(19) United States
(12) Patent Application Publication
(10) Pub. No.: US 2023/0360772 A1
(43) Pub. Date: Nov. 9, 2023

(54) VIRTUAL REALITY BASED COGNITIVE THERAPY (VRCT)

(71) Applicant: Penumbra, Inc., Alameda, CA (US)

(72) Inventors: Marguerite Manteau-Rao, Palo Alto, CA (US); William Ka-Pui Yee, Alameda, CA (US)

(21) Appl. No.: 17/736,592

(22) Filed: May 4, 2022

(51) Int. Cl. G16H 20/70; G16H 40/67; G16H 10/20; G06T 13/40; G06T 19/00

(52) U.S. Cl. CPC G16H 20/70; G16H 40/67; G16H 10/20; G06T 13/40; G06T 19/006

(57) ABSTRACT

Systems and methods of virtual reality (VR) may be used with Cognitive Therapy (CT) to help people experiencing mental health issues stemming from, e.g., automatic thoughts and associated feelings. VRCT can immerse a patient in a virtual world and allow a patient's avatar to interact with virtual, non-playable characters, to reduce reliance on live therapists and improve availability and engagement. Patients may virtually meet with an avatar of a virtual therapist, who will guide a patient to recognize intense negative and/or automatic thoughts about a situation and work towards a more realistic response to the situation. A therapist avatar communicating in a VR world may prompt a patient to discuss a recent situation associated with strong thoughts and emotions in order to help the patient, e.g., catch one or more automatic thoughts, check the accuracy of the automatic thoughts, and change the automatic thoughts to something more accurate and/or less intense.

### BACKGROUND OF THE DISCLOSURE

The present disclosure relates generally to virtual reality (VR) systems and more particularly to providing virtual reality Cognitive Therapy (VRCT) or therapeutic activities or therapeutic exercises to engage a patient experiencing one or more cognitive-related mental or behavioral health disorders.

### SUMMARY OF THE DISCLOSURE

Virtual reality (VR) systems may be used in various medical and mental health-related applications including Cognitive Therapy. VR Cognitive Therapy as described in this disclosure is based on the way individuals perceive a situation that is more closely connected to their reactions than to the situation itself. In other words, the individuals' perceptions are often distorted and unhelpful in a particular situation, especially when they are distressed. The methods of VR Cognitive Therapy as described in this disclosure are used to assist people or patients to identify distressing thoughts and evaluate how realistic those thoughts are. The methods then assist the users or patients to change their distorted thinking. With a more realistic assessment of a particular situation, the users or patients can overcome their misperceptions and misplaced reactions, which can lead to improved thoughts and improved emotional states.

### BRIEF DESCRIPTION OF THE DRAWINGS

FIG. 1 illustrates the VR Cognitive Therapy model in accordance with some embodiments of the present disclosure;

FIG. 2 illustrates exemplary “Catch It,” “Check It,” and “Change It” process as applied to a VR Cognitive Therapy model in accordance with some embodiments of the present disclosure;

FIG. 3 illustrates exemplary “Catch It” process as applied to a VR Cognitive Therapy model in accordance with some embodiments of the present disclosure;

FIG. 4 illustrates exemplary “Check It” process as applied to a VR Cognitive Therapy model in accordance with some embodiments of the present disclosure;

FIG. 5 illustrates exemplary “Change It” process as applied to a VR Cognitive Therapy model in accordance with some embodiments of the present disclosure;

FIG. 6A illustrates exemplary challenges and problems with conventional methods (such as writing in a ledger) in traditional Cognitive Therapy, in accordance with some embodiments of the present disclosure;

FIG. 6B depicts a chart with exemplary challenges of traditional Cognitive Therapy, in accordance with some embodiments of the present disclosure;

FIG. 7 illustrates exemplary components of a VRCT system, including biometric sensors, in accordance with some embodiments of the present disclosure;

FIG. 8 illustrates a flow-chart for an exemplary “Catch It” process as applied to a VR Cognitive Therapy model, in accordance with some embodiments of the present disclosure;

FIG. 9 illustrates a flow-chart for an exemplary “Check It” process as applied VR Cognitive Therapy model, in accordance with some embodiments of the present disclosure;

FIG. 10 illustrates a flow-chart for an exemplary “Change It” process as applied VR Cognitive Therapy model, in accordance with some embodiments of the present disclosure;

FIG. 11 is an illustrative depiction of a user interface, e.g., a virtual therapist's office, for a VR therapy platform, in accordance with some embodiments of the disclosure;

FIG. 12 is an illustrative depiction of a user interface for an exemplary portion of the “Catch It” exercises, e.g., at a lake with a virtual therapist, for a VR therapy platform, in accordance with some embodiments of the disclosure;

FIG. 13 is an illustrative depiction of a user interface for an exemplary portion of the “Check It” exercises, e.g., a virtual therapist's office, for a VR therapy platform, in accordance with some embodiments of the disclosure;

FIG. 14 is an illustrative depiction of a user interface for an exemplary portion of the “Check It” exercises, e.g., a virtual therapist's office, for a VR therapy platform, in accordance with some embodiments of the disclosure;

FIG. 15 is an illustrative depiction of a user interface for an exemplary portion of the “Check It” process, e.g., a virtual therapist's office, for a VR therapy platform, in accordance with some embodiments of the disclosure;

FIG. 16 is an illustrative depiction of a user interface for an exemplary portion of the “Change It” process, e.g., a virtual therapist's office, for a VR therapy platform, in accordance with some embodiments of the disclosure;

FIG. 17 is an illustrative depiction of a user interface for an exemplary portion of the “Change It” process, e.g., a virtual therapist's office, for a VR therapy platform, in accordance with some embodiments of the disclosure;

FIG. 18A illustrates a flow-chart for an exemplary process for collecting biometric feedback as applied VR Cognitive Therapy model, in accordance with some embodiments of the present disclosure;

FIG. 18B is an illustrative chart for collected biometric feedback as applied VR Cognitive Therapy model, in accordance with some embodiments of the present disclosure;

FIG. 18C illustrates a flow-chart for an exemplary process for collecting biometric feedback in a VR therapy platform, in accordance with some embodiments of the present disclosure;

FIG. 19A is a diagram of an illustrative system of a VR Cognitive Therapy platform, in accordance with some embodiments of the disclosure;

FIG. 19B is a diagram of an illustrative system of a VR Cognitive Therapy platform, in accordance with some embodiments of the disclosure;

FIG. 20 is a diagram of an illustrative system of a VR Cognitive Therapy platform, in accordance with some embodiments of the disclosure;

FIG. 21 is a diagram of an illustrative system of a VR Cognitive Therapy platform, in accordance with some embodiments of the disclosure; and

FIG. 22 is a diagram of an illustrative system of a VR Cognitive Therapy platform, accordance with some embodiments of the disclosure.

### DETAILED DESCRIPTION OF THE DISCLOSURE

Various systems and methods disclosed herein are described in the context of a VR therapeutic system for helping patients, but the examples discussed are illustrative only and not exhaustive. A VR system as described in this disclosure may also be suitable for coaching, training, teaching, and other activities. Such systems and methods disclosed herein may apply to various and many VR applications. In some embodiments, a VRCT platform may comprise one or more VR applications. In some embodiments, a VRCT platform may comprise one or more automatic speech recognition system and natural language processing applications as well as biometric sensing, recording, and tracking systems for building biometric models for comparisons, diagnostics, recommendations for, e.g., treatment and/or intervention, etc.

In the context of the VRCT system, the word “patient” may generally be considered equivalent to a subject, user, participant, student, etc., and the term “therapist” may generally be considered equivalent to doctor, psychiatrist, psychologist, psychotherapist, physical therapist, clinician, coach, teacher, social worker, supervisor, or any non-participating operator of the system. A real-world therapist may configure the system and/or monitor via a clinician tablet, which may be considered equivalent to a personal computer, laptop, mobile device, gaming system, or display.

Some embodiments may use a “virtual therapist” and/or a “therapist avatar,” which may be used interchangeably herein. As part of a VRCT platform, a virtual therapist may comprise (and/or work in conjunction with) a virtual assistant and automatic speech recognition (ASR) service working in conjunction with a natural language processing (NLP). A therapist avatar may be considered an on-screen avatar of a virtual therapist. In some embodiments, other non-playable avatars may be controlled by a virtual therapist and/or a VRCT platform and feature a different appearance, voice, and/or other virtual characteristics.

Some embodiments may include a digital hardware and software medical device that uses VR for health care, focusing on mental, physical, and neurological rehabilitation, including various biometric sensors, such as sensors to measure and record heart rate, respiration, temperature, perspiration, voice/speech (e.g., tone, intensity, pitch, etc.), eye movements, facial movements, jaw movements, hand and feet movements, neural and brain activities, etc. For instance, voice biomarkers and analyzers may be used to assess and track emotional states and/or determine intensity values for emotions.

The VR device may be used in a clinical environment under the supervision of a medical professional trained in rehabilitation therapy. In some embodiments, the VR device may be configured for mental health, behavioral health, mindfulness, and/or wellness applications, including personal therapeutic use at home. In some embodiments, the VR device may be configured for remote sessions and remote monitoring. A therapist or supervisor, if needed, may monitor the experience in the same room or remotely. In some cases, a therapist may be physically remote or in the same room as the patient. Some embodiments may require someone, e.g., a nurse or family member, assisting the patient to place or mount the sensors and headset and/or observe for safety. Generally, the systems are portable and may be readily stored and carried. In some embodiments, a VR device may be used independently by a patient or user, e.g., without a therapist present virtually or physically. For instance, independent use may be required as “homework” between other guided therapy sessions.

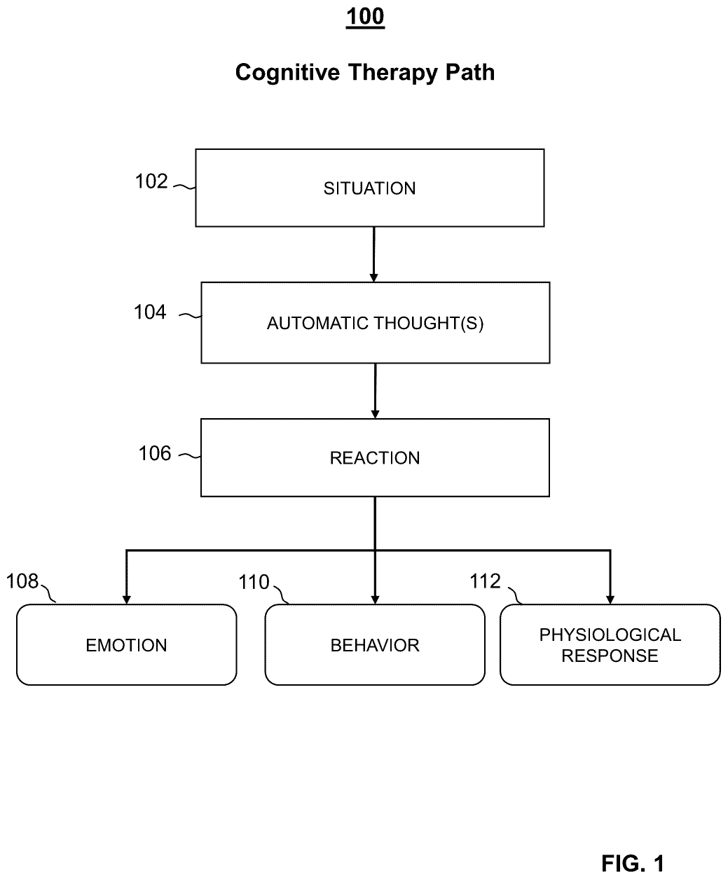

Cognitive Therapy as described in this disclosure may be used to treat patients with a range of mental health disorders, most notably depression. Other indications include anxiety, substance abuse, insomnia, chronic pain, migraine, gastro-intestinal disorders, eating disorders, etc. The Cognitive Therapy model, as illustrated in FIG. 1, describes how thoughts and perceptions influence feelings and behaviors. As indicated in this Cognitive Therapy model, a person encounters a situation and he or she may respond with certain biological states and automatic thoughts. Those thoughts may trigger certain reactions. The reactions can be generally categorized as emotional, behavioral, and/or physical reactions.

Mental health disorders can impede a person's quality of life. A change in thinking or a particular way of thinking is a key feature of depression, and these thoughts often reflect a change in the way a person with depression has come to think about themselves. For example, a devoted parent may believe they are doing a terrible job raising a child. A competent employee may view himself or herself as a failure. A person learning how to identify what he/she is thinking can be an important step in reducing depression. Cognitive Therapy may begin with teaching a person to notice when his/her mood has changed or intensified in a negative direction. One might also notice behaviors associated with negative thinking such as avoidance and/or engaging in unhelpful behaviors (e.g., sleeping too much or overeating). When either mood has changed in a negative direction or a person is engaging in an unhelpful or unhealthy behavior, Cognitive Therapy suggests asking the cardinal question of Cognitive Therapy: “What was just going through my mind?” This is an important approach to identify automatic, unhelpful thoughts. It assists and guides people to pay special attention to thoughts that can get in the way of or prevent them from taking the necessary steps to achieve what is most important to them. People with depression or other forms of mental health disorders tend to make consistent errors in their thinking. Identifying and labeling thinking errors is an important step in gaining perspective and applying Cognitive Therapy.

Mental illness can cause those affected to perceive a situation in a way that is disjointed from the facts or reality of the situation itself, resulting in thinking errors. Thinking errors are self-defeating or self-deprecating patterns of thinking that do not accurately correspond to reality or arriving at the root cause, and as such, can cause a patient to become lost in his or her negative attitude toward himself/herself. For example, a young adult with body dysmorphia and/or an eating disorder may see herself as being overweight and/or unattractive despite being healthy. As a result, she may begin to starve herself and/or overly exercise as a result of anorexia, or she may become bulimic and force herself to throw up what she eats. Mental health disorders can create negative thoughts and poor emotional states, and which can potentially result in negative physical repercussions.

Identifying and labeling these “thinking errors” can help someone gain perspective. For example, suppose being of service to one's family is a strong value of a patient. For example, a grandmother does what she can to help her grandchildren, but at times she is not available. She might have the (automatic) thought, “I'm a failure as a grandparent,” which is likely an incorrect assumption. There are many forms of such “thinking errors” for people experiencing mental health disorders. Some of the thinking errors may include:
All-or-nothing thinking—View a situation in only two categories instead of on a continuum. For example, “If I'm not a total success, I'm a failure.”
Catastrophizing—Predict the future negatively without considering other, more likely, or just as likely outcomes. For example: “I'll be so upset, I won't enjoy myself at all.
Disqualifying or discounting the positive—Telling yourself that positive experiences, deeds, or qualities don't count. For example: “I did a good job on the project but that doesn't mean I'm good at my job; I just got lucky.”
Emotional reasoning—Thinking that something must be true because it “feels” true. For example: “I know I do a lot of things OK at work, but I still feel incompetent.”
Labeling—Putting a fixed, global label on yourself or others without considering evidence that might lead to a less extreme conclusion. For example: “I'm a total loser.”
Magnification/Minimization—When evaluating yourself, another person, or a situation, you unreasonably magnify the negative and/or minimize the positive. For example: “Getting a mediocre grade proves how stupid I am.”
Mental Filter—Paying a great deal of attention to one negative detail instead of looking at the whole picture. For example: “Because I got one negative comment on my evaluation (which may also contain several excellent comments), it means I'm doing a lousy job.”
Mind-reading—Believing you know what others are thinking and failing to consider other, more likely or just as likely possibilities. For example: “They're thinking I don't know what I'm doing.”
Overgeneralization—Making a sweeping negative conclusion that goes far beyond the current situation. For example: “Because I felt uncomfortable at the meeting, I don't have what it takes to work here.”
Personalization—Believing others are behaving negatively because of you, without considering more reasonable explanations for their behavior. For example: “My neighbor didn't say hello to me because I did something to upset them.”
Should and Must statements—Having a precise, fixed idea of how you or others should behave. For example: “I shouldn't make any mistakes.”
Tunnel Vision—Only see the negative aspects of a situation. For example: “The whole day was terrible.” The negative thought did not take into consideration the positive feeling after getting dressed, cleaning up the kitchen, going for a walk, and/or talking to a friend on the phone.

In responding to automatic thoughts, most people with mental health disorder, such as depression, believe that the situations in their lives cause their sadness. While life includes many trying and difficult situations, feelings are derived from what we think about and how we interpret the situations that we face. It is not the situations in our lives that cause distress, but rather our interpretations of those situations. Part of the cognitive approach as enabled on a VR platform as described in this disclosure comprises of consideration of the situation, the emotions felt, and the thoughts associated with that situation. Most people are generally aware of how they generally feel in a situation, e.g., “feeling good” or “not feeling good.” Some people may be aware of, e.g., an emotional response and an associated emotional state. For example, suppose you texted a close friend several hours ago and they didn't text back. You might have the automatic thought, “They don't want to spend time with me anymore.” This thought would likely lead you to feel sad and dejected. Now, imagine you had the thought, “Something is wrong.” You might feel anxious. Once you understand what you are thinking, how you feel makes sense.

Expanding on this cognitive approach, as facilitated on a VR platform, the method involves Socratic Questioning. Asking the right questions may illuminate the reason or rationale for the automatic thoughts and associated feelings. Socratic Questioning includes:
What was just going on through my mind?
What makes me think this thought is true? Is there any evidence it might not be true, or not completely true?
Is there another way of looking at this situation?
If the worst happens, what could I do? What's the best that could happen? What is most likely to happen?
What's the effect of believing this thought? What could happen if I changed my thinking?
What would I tell my friend [think of a specific person] if they were in this situation and had this thought?
What can I do about this now?

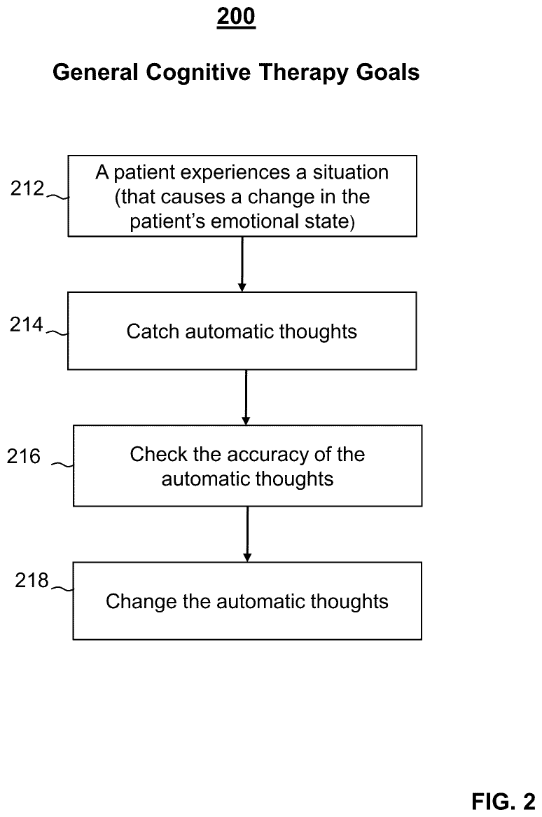

These Socratic Questions will help to evaluate negative automatic thoughts in a more reasonable, balanced way and develop responses that are more helpful. By answering these Socratic Questions with more reasonable, balanced responses, one may find life can be experienced more realistically and beginning to feel better. Hence, the method of VR Cognitive Therapy as described in this disclosure comprises of the steps of “Catch it,” “Check it,” and “Change it,” as illustrated in FIG. 2.

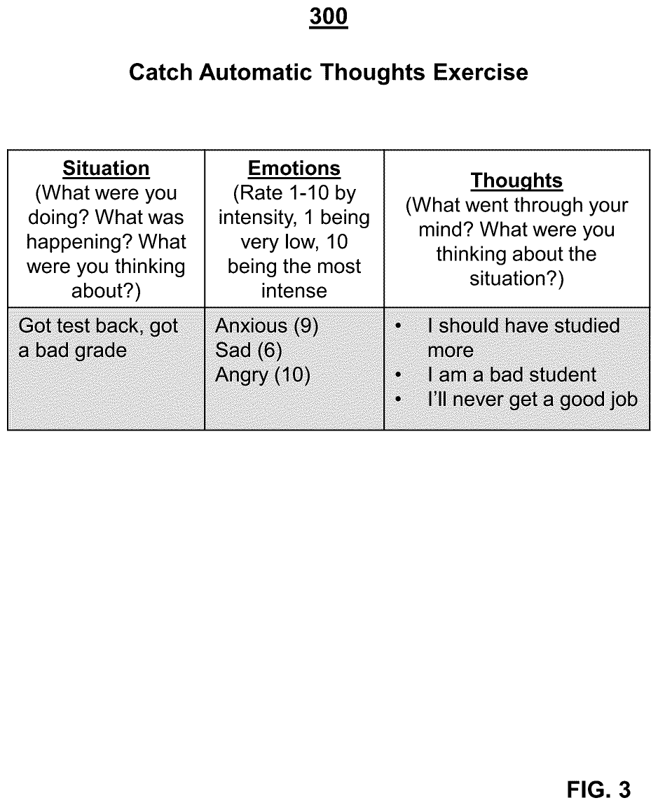

The “Catch It” step involves catching the automatic thoughts. FIG. 3 illustrates some of the details involved in the “Catch It” step. “Catch It” involves catching the automatic thought associated with a change in mood. Sometimes it is easier to identify a shift in mood first and then to ask yourself what was going through your mind just then. For example, patients may be coached to think about a time in the recent past when they noticed a negative shift in their mood. For some patients, it may be helpful to imagine or describe the situation that led to the negative mood state. Then, patients can be asked to identify the automatic thought associated with the mood change.

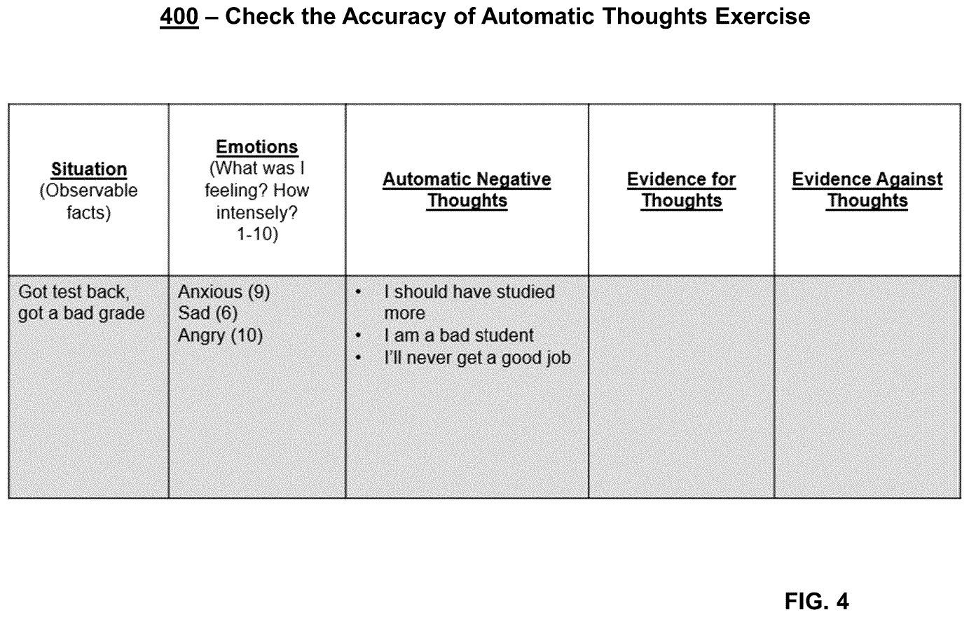

The “Check It” step involves checking the automatic thoughts for accuracy. FIG. 4 illustrates some of the details involved in the “Check It” step. For “Check It,” patients are instructed to check or evaluate whether the thought is true, complete, or balanced. Patients may ask themselves, “What is the evidence indicating that the thought is true?” In some cases, patients may be instructed to ask themselves whether they think the thought is complete or balanced. A complete thought is based on all of the important and relevant information related to the situation that was associated with the initial automatic thought. A balanced thought includes information that is not extreme and is fairer and more reasonable than the initial automatic thought.

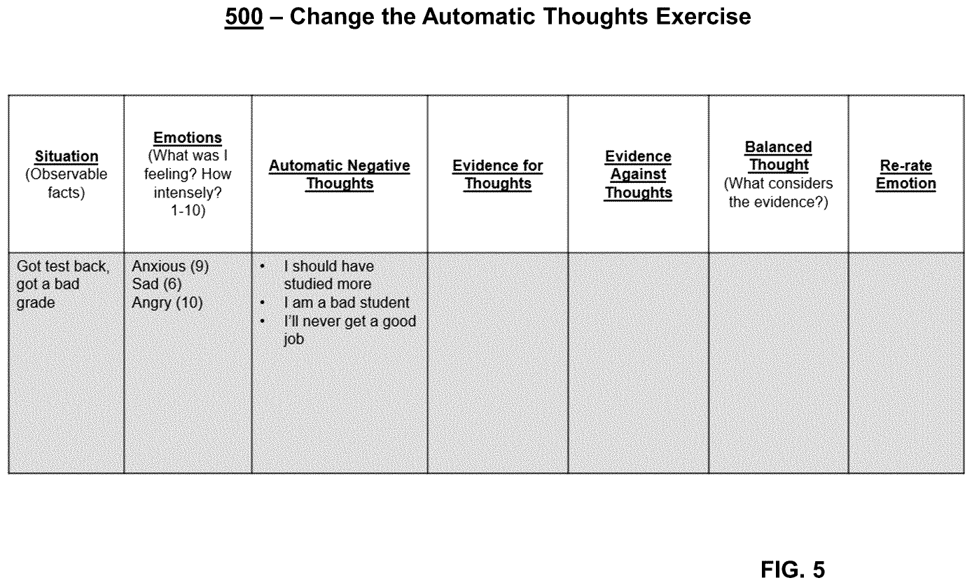

The “Change It” step involves changing the automatic thought into a more accurate thought. FIG. 5 illustrates some of the details involved in the “Change It” step. For “Change It,” patients are instructed to think of a replacement thought that is true, complete, or more balanced than the initial automatic thought. For example, instead of putting yourself down in a harsh, condemning way, talk to yourself in the same compassionate way you would talk to a friend with a similar problem

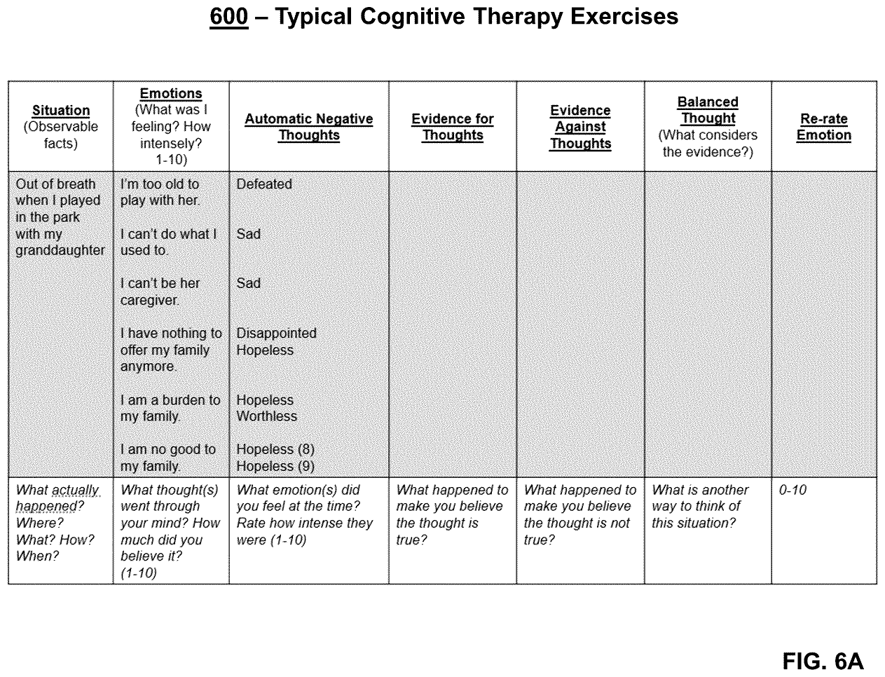

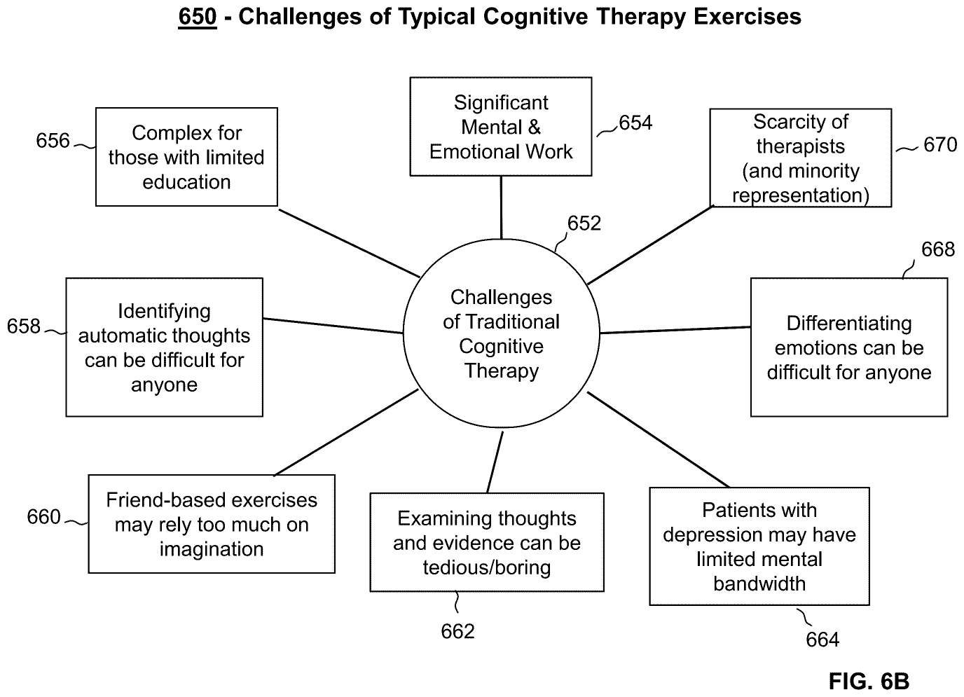

While Cognitive Therapy can be effective, it requires a lot of mental work with patients. It is an intellectual exercise that can be challenging for some people with limited education or ability for insights. People with depression or other mental health problems or challenges may have limited mental capacity or bandwidth and energy to effectively use Cognitive Therapy due to their condition. Identifying automatic thoughts can be challenging for anyone. Examining the thoughts and evidence behind them can be tedious, straining, and stressful. FIGS. 6A and 6B illustrate examples of challenges and problems of typical Cognitive Therapy. As illustrated in FIG. 6A, the process would require filling out a worksheet or writing in a notebook and identifying a situation, the associated automatic thoughts, and resulting emotions and/or mood. The process would then require the examination of evidence to support the automatic thoughts and evidence that does not support those thoughts. The process would then further identify and examine possible alternative thoughts, and finally, rate the mood after having completed this Cognitive Therapy process. Such a process has several drawbacks, as depicted in chart 650 in FIG. 6B, including, chiefly, that filling out the details on a CT ledger workbook page can often feel tedious, boring, and overwhelming. Even when using a computerized ledger, the process can be tiresome and solitary. Cognitive Therapy, e.g., as performed traditionally, is not as accessible as it could be and the tedious processes may, in fact, contribute to issues with follow-through and continuity. If the goal is to have more patients participate in, engage with, and follow through with Cognitive Therapy, then many off-putting challenges must be addressed.

Chart 650 of FIG. 6B depicts several exemplary challenges with typical (traditional) Cognitive Therapy 652 exercises, such as asking a patient to fill out a ledger worksheet of FIG. 6A. As identified in box 654, Cognitive Therapy may require significant mental and emotional work that may wear out patients, cause anxiety for patients, and/or feel overwhelming for patients. Similarly, traditional CT may be complex and difficult for patients who may have limited education, as seen in box 656. Box 658 describes how identifying automatic thoughts and emotions can be difficult for anyone sometimes, regardless of educational background. Emotions may make clear thinking difficult. For instance, in some cases, a patient may identify a feeling of frustration or anger but not necessarily connect, e.g., a particular situation or an automatic thought or feeling that came from the situation. Differentiating emotions, as seen in box 668, can be difficult on anyone. For example, discerning frustration from anger from rage may not be apparent to one experiencing the emotions. In some cases, as seen in box 664, patients experiencing certain emotional conditions may have limited bandwidth to go through the steps of a ledger worksheet by themselves, or even a guided by a therapist. Examining thoughts and evidence, as stated in box 662, can be tedious and/or boring in traditional Cognitive Therapy. Moreover, the environment, such as a therapist's office or alone in one's room, can be perceived as tedious, boring, distracting, intimidating, and/or otherwise discouraging. Certain exercises, such as those depending on changing perspectives and having an imaginary conversation with a friend, described in box 660, may be too reliant on imagination. For instance, requiring an imaginary conversation may not deeply engage a patient or, in some cases, visualizing such a conversation may be too stressful or mentally taxing.

Another key downside of traditional Cognitive Therapy is the fact that, as depicted in box 670 of FIG. 6B, there exists a scarcity of real-world therapists able to help guide Cognitive Therapy, specifically for therapists from and working with underrepresented groups and minorities. Such a shortage of therapists often requires significant homework and self-analysis by patients. Patients may be in locations with a dearth of trained therapists. In cases where a therapist may actually be available, a patient's potential comfortability with a therapist may be unfortunately sacrificed due to insufficient representation. For instance, a minority patient may not get to experience an actual therapist from a similar ethnicity, race, and/or background and would put the patient more at ease during the exercises. Likewise, a patient might not follow through with therapy because of an unavailability to hear their own language or accent. Some patients may be more comfortable with and/or engaged by therapists who share significant pieces of their own identity, such as characteristics like identified gender, orientation, race, religion, age, height, weight, and/or appearance, e.g., hair style and clothing style. Patients should have options for therapists who make them comfortable.

As one can appreciate, Cognitive Therapy can be a challenging and laborious process. This disclosure describes the opportunity to use VR to remove some of the engagement barriers in Cognitive Therapy exercises which can lead to better adherence to treatment and improved health outcomes. Not only might VR therapy help compensate for an insufficient number of trained professionals, but customizable virtual avatars may also help fill the gaps of underrepresented groups and minorities in a therapy-related profession(s). Moreover, virtual avatars can shoulder the burdens of structure by requesting information and prompting patients to listen and consider. Receiving input from a patient via the virtual platform can help minimize a patient's thought and effort required by, e.g., filling out a worksheet or notebook. What may have seemed like significant mental and emotional work for a worksheet alone may feel like a fun VR activity with a familiar therapist in an engaging virtual world. User interface options may help exercises such as identifying initial thoughts and/or differentiating emotions felt. A customizable friend avatar can encourage conversation and inspire compassion.

Traditional Cognitive Therapy has initial drop-out at sixteen percent or higher even before the treatment is started. Even after getting beyond the initial drop-out of patients, studies have shown that for those who have started the traditional Cognitive Therapy, another twenty-six percent (almost a third of those tried the treatment) of the patients drop out shortly thereafter. That is almost fifty percent of patients dropping out at the start of therapeutic treatment.

As disclosed herein, a VR Cognitive Therapy platform can help reduce patient feelings about therapy being tedious, boring, overwhelming, and/or complicated. No longer “alone,” in some embodiments, a VR Cognitive Therapy platform can present a virtual therapist avatar that may guide a patient through one or more VRCT activities such as “Catch It,” Check It,” and/or “Change It” exercises. VR activities offer an appealing world that keeps a patient's focus and promotes progress. A VR Cognitive Therapy platform can help improve engagement, boost retention, reduce drop-out, and promote therapy continuity. In some embodiments, a VR Cognitive Therapy platform can engage a patient in Cognitive Therapy while measuring and monitoring biophysical traits that may indicate progress in the short-term or long-term. Customizable avatars for users, virtual therapists, and virtual friends offer an engaging way to make patients feel more comfortable. When a patient's emotional state is not optimal, therapeutic help may only be as far away as putting on an HMD and beginning a VRCT session.

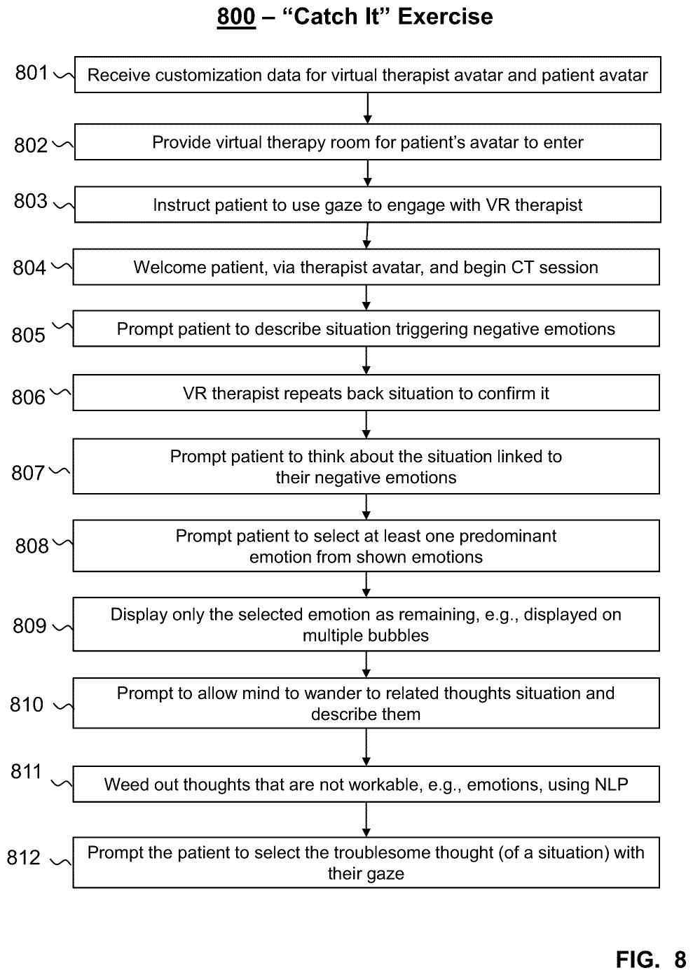

The Cognitive Therapy session starts with the “Catch It” exercise in which a detailed example is illustrated in FIG. 8. First, before starting the Cognitive Therapy session, the user or patient can select or construct a customized virtual reality environment or space for the session. The user or patient is immediately involved or engaged even before the actual therapy begins. For example, the user can choose an office setting or an outdoor setting for the Cognitive Therapy session. The user can choose size of the office, the color scheme, the lighting, the furniture, etc. that would be most comfortable for him or her. Alternatively, the user can choose an outdoor setting such as a park or beach as the place for the Cognitive Therapy session. In addition, the user can choose various background features, such as nature scenes, background sounds, lighting, etc. Perhaps more importantly, as illustrated in Step 801 of FIG. 8, the user can select or create an avatar therapist for the Cognitive Therapy session. The user can select the age, gender, skin color, hair color, hair style, clothes, voice, weight, height, and/or any other characteristics for an avatar therapist to create the most comfortable engagement for him or her. In this example of a “Catch It” exercise, as Step 802, the patient enters the virtual therapy room, and the patient can see the customized therapist avatar that he or she created. Initially, the patient may see the therapist avatar has their hands resting on their lap. A position or posture that is considered as most relaxed, none threatening, or most neutral.

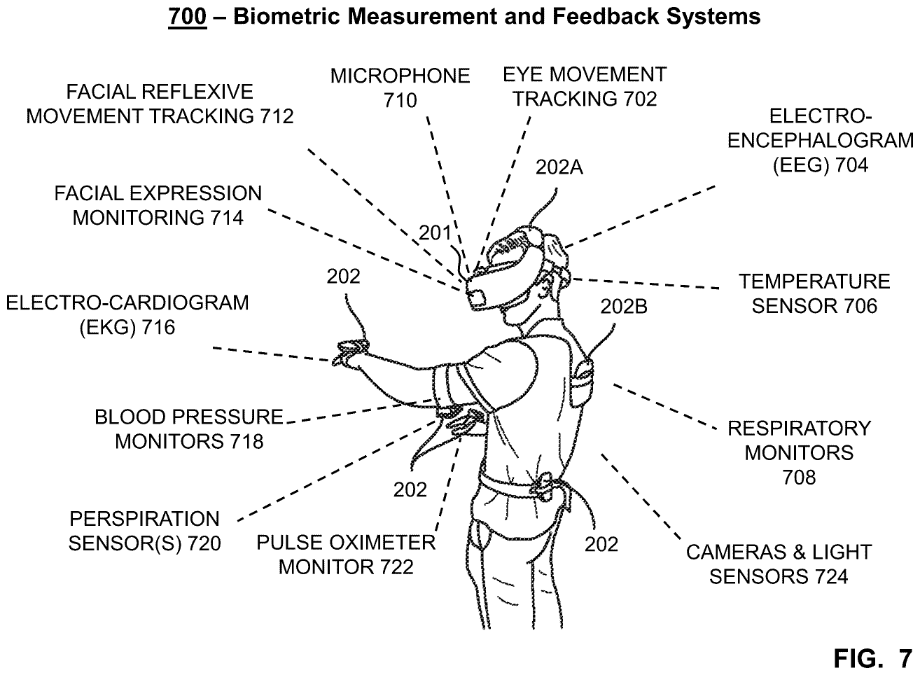

Concurrently, as the user or patient starts or enters in the VR environment, biometric sensors start to measure and record biometric data of the patient for building biometric models for comparisons, diagnostics, and recommendations. For example, the initial biometric data may be used to build a baseline biometric model for comparison to data collected throughout the Cognitive Therapy session and especially for comparison at the end of the session. In addition, the collected data may be analyzed for various diagnoses as well as for recommendations for future activities, exercises, treatments, etc. In some cases, a patient may not be fully aware of how they are feeling. In some cases, a patient may perceive that they are not feeling good but may have difficulty identifying, e.g., more specifically how they feel until some biometric data, such as blood pressure or heart rate, is shown to them. FIG. 7 depicts a VR system with exemplary components of a VRCT platform including several biometric sensors. Some embodiments may include sensors, such as eye movement tracking 702, electroencephalogram (EEG) 704, temperature sensor 706, respiratory monitors 708, microphone 710, facial reflexive movement tracking 712, facial expression monitoring 714, electrocardiogram (EKG) 716, blood pressure monitors 718, perspiration sensor 720, pulse oximeter monitor 722, and cameras and light sensors 724. The biometric sensors measure and record a variety of biometric data including heart rate, respiration, temperature, perspiration, voice/speech (e.g., tone, intensity, pitch, etc.), eye movements, facial movements, mouth and jaw movements, hand and feet movements, neural and brain activities, etc., throughout the Cognitive Therapy session.

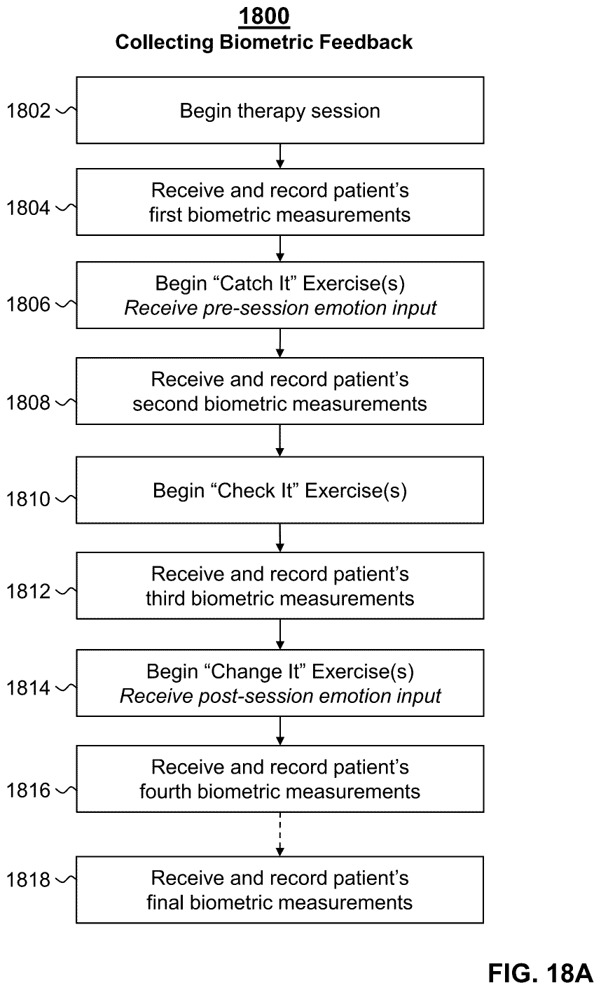

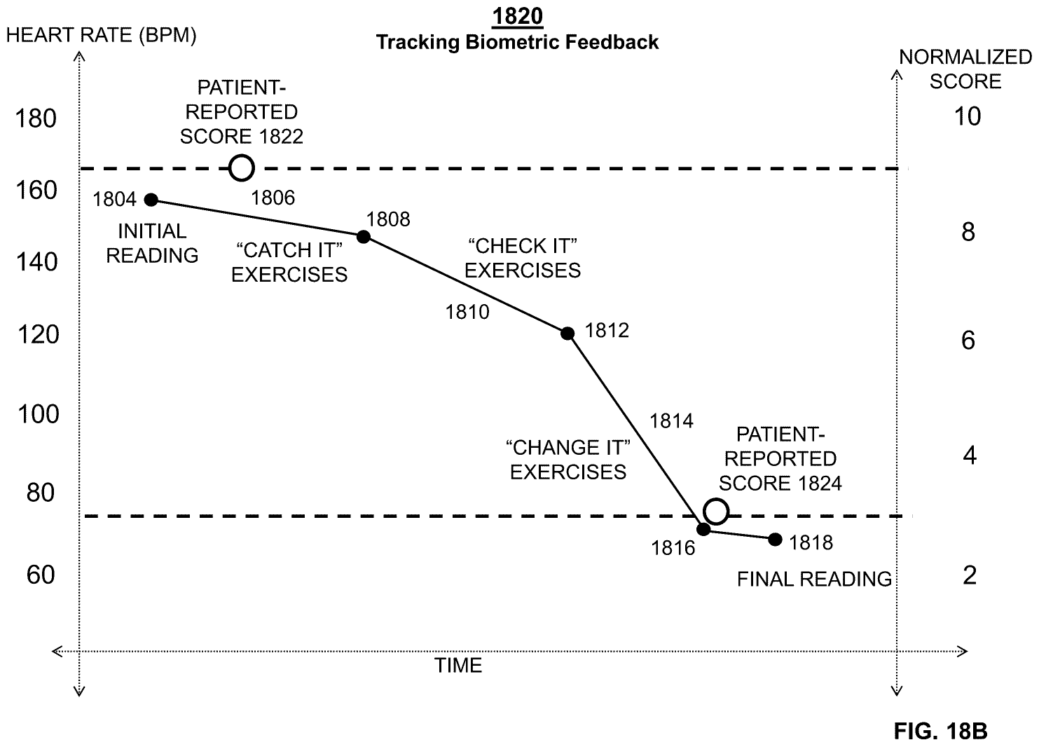

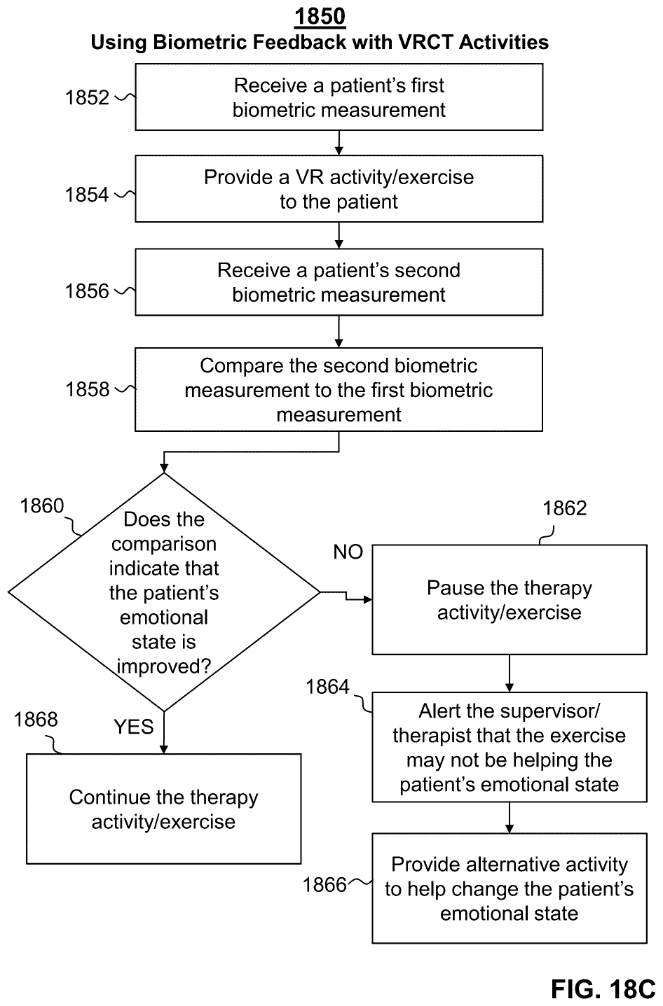

In some embodiments, biometric data may be used to correlate with the state of emotional wellness of the patient at the start of the Cognitive Therapy session, throughout the exercises, and at the end of the session. For example, a therapist and/or patient may be able to help differentiate emotional feelings or emotional states on a spectrum such as, e.g., feelings of depression, anxieties, frustrations, anger, rage, etc. In some embodiments, with a helpful Cognitive Therapy session, a chart like FIG. 18B may track biometric data depicting a patient calming down or improving emotional state, e.g., experiencing less intensity for one or more emotions and/or thoughts over the session. In some embodiments, therapy exercises may affect a patient and her biometric data differently, but the end goal of VRCT will be to achieve a measurement indicating that the exercises together will improve a patient's emotional state and, e.g., calming the patient, reducing the state of depression, anxiety, frustration, anger, rage, etc. For instance, a decline in a biometric indicating improvement in a patient's emotional state, emotional conditions, and associated bio-physical states, conditions, and/or markers—e.g., lower perspiration, lower heart rate, lower blood pressure, improved respiration, fewer involuntary movements, etc.—may not be achieved until after the final exercise, e.g., the “Change It” exercise(s) where a patient may receive rational, calm feedback from a virtual friend or therapist about a problematic situation. An exemplary process for collecting biometric feedback, e.g., in Cognitive Therapy, is depicted as process 1800 in FIG. 18A, and an exemplary process for capturing and comparing biometric measurements for a patient with a patient's input, e.g., in Cognitive Therapy, is depicted as process 1850 in FIG. 18C.

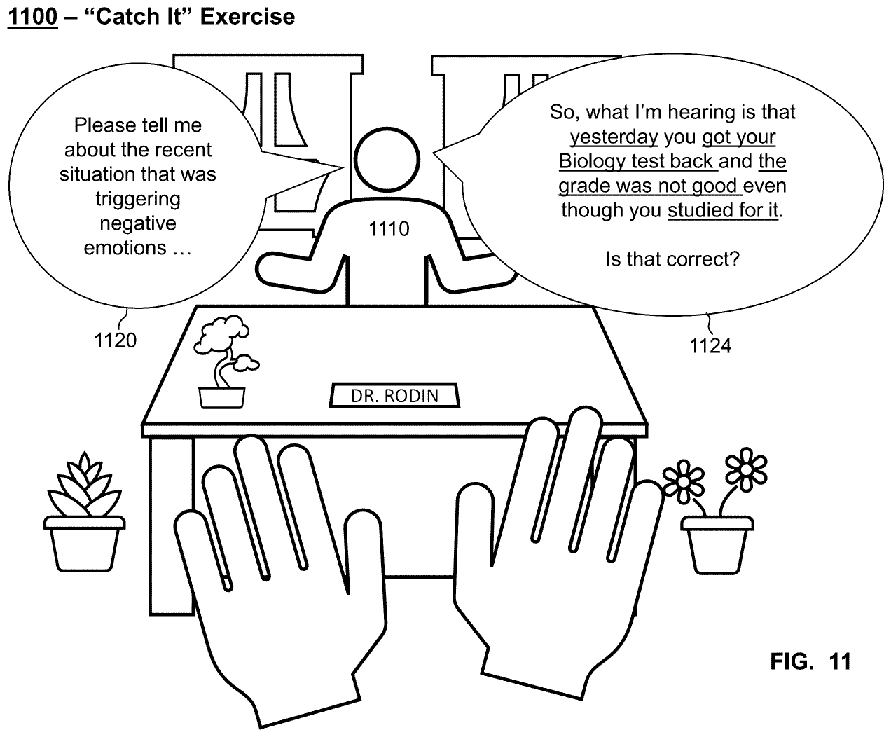

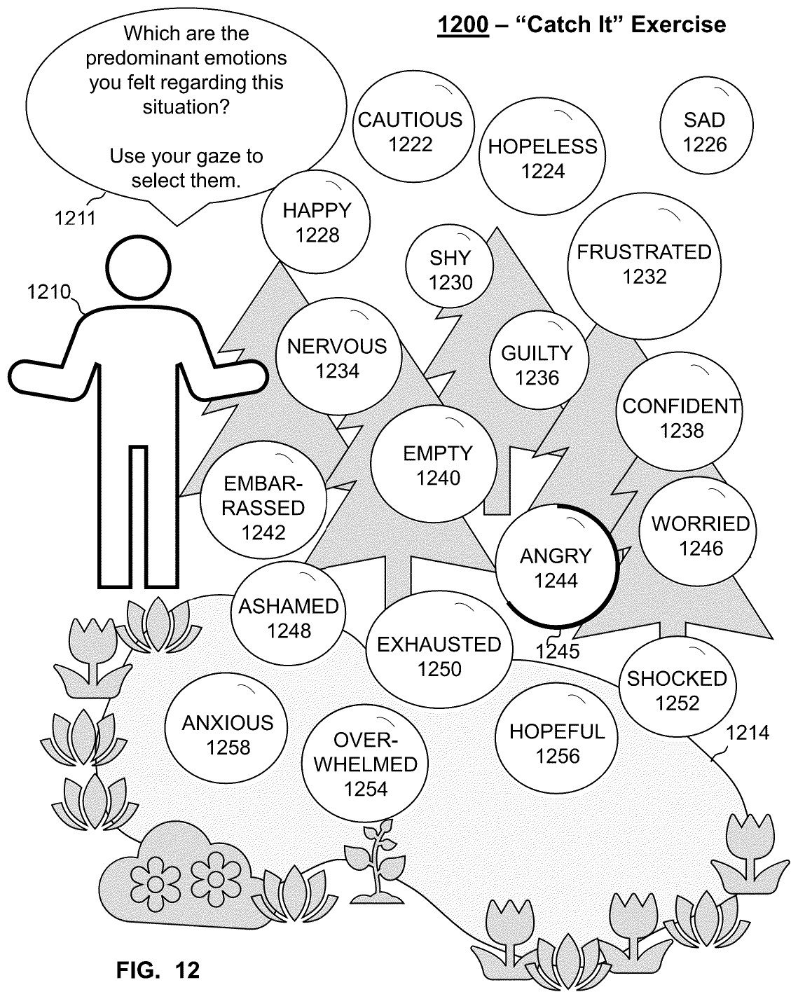

Continuing with the “Catch It” exercises, to facilitate engagement and to simulate greeting gestures, the patient is instructed by the VR Cognitive Therapy program to raise their hands in front of the VR head mounted display (HMD) and move them. In response, the patient can see the therapist avatar mirroring the movement, as depicted in FIG. 11. After the virtual avatar therapist enters the room and takes a seat across from the patient, the VR Cognitive Therapy program instructs the patient to use their gaze or voice to activate the Cognitive Therapy program to start the engagement with the virtual or VR therapist avatar, in Step 803 of FIG. 8. The VR therapist welcomes the patient and proposes to start the Cognitive Therapy session related to a situation that is triggering negative emotions, in Step 804. The therapist, in Step 805, invites the patient to describe out loud the situation. With the patient's description, the avatar reflects and/or repeats back on what was heard to the patient and the therapist asks the patient to confirm. After the patient answers “yes” and they have the situation in mind, in Step 806, the VR therapy may take the patent to a select environment for the next step of the Cognitive Therapy exercise. The selected VR environment may be one that would promote mindful exercises with mindful inquiries, such as a lake nature environment. This mindful inquiry exercise may be a pre-determined or pre-recorded guided practice that is broken down into strings, segments, or sets to match the pace of inquiry for the patient. Accordingly, the mindful exercise may be tailored to match the particulars of the patient. During the mindful inquiry, in Step 807, the VR therapy invites the patient to think about the situation that is linked to their negative emotions. The therapist then asks, in Step 808, the patient to use their gaze or voice to select at least one predominant emotion from different emotions that are shown in the VR environment (e.g., bubble prompts floating above the table in the office environment or bubble prompts floating up from a surface of the lake, as shown in FIG. 12).

For instance, in FIG. 12, emotions 1222-1258 as depicted in scenario 1200 may appear to bubble from lake 1214. Such emotions may include, e.g., cautious 1222, happy 1228, sad 1226, shy 1230, frustrated 1232, empty 1240, embarrassed 1242, angry 1244, worried 1246, anxious 1258, overwhelmed 1254, hopeful 1256, guilty 1236, nervous 1234, shocked 1252, and more. To select an emotion with gaze, in some embodiments, a cursor may be moved based upon gaze (e.g., HMD movement and/or eye tracking) and a bubble may be selected by holding the gaze until a time tracker fills up. For instance, time tracker 1245 depicts, e.g., 4 seconds of a 6-second time tracker, has filled up based on a held gaze for angry 1244 bubble. In some embodiments, the patient may select one or more emotions using speech, gaze, and/or by pointing at the emotion bubble or cloud icon with their hands. In some embodiments, a patient may be asked to speak selected emotions aloud for audio capture. Scenario 1200A depicts that one or more bubbles or clouds may be selected; however, some embodiments may use only one selected bubble. Bubbles may be considered representative icons or shapes used for emotions, and other icons or shapes may be substituted; however, the extended analogy of emotions “bubbling up” in scenario 1200A may prove to make patients more relaxed and/or engaged.

Back to process 800 of FIG. 8, in the VR environment, in Step 809, only the selected emotion remains and is displayed on multiple bubbles. The therapist asks the patient to verbalize, say, and rate the intensity level of their emotion, for example from a scale of 1 to 10. With that input, the emotion bubbles may change color to reflect the emotional intensity level of the patient. The bubble color intensity may use bright red to represent intense anger. Other colors and brightness of the colors may be used to reflect emotional intensity level. Next, the therapist invites the patient to allow their mind to wander into thoughts related to the situation and describe them as they arise, in Step 810. The spoken thoughts then appear or materialize in virtual clouds in the VR environment. The therapist then helps to weed out thoughts that are not workable or applicable, e.g., thoughts that are expression about emotions (using natural language processing (NLP)) technology, in Step 811. Finally, in this example, the therapist asks the patient to select the troublesome thought (of a situation) with their gaze, in Step 812. Only that identified thought remains in a cloud.

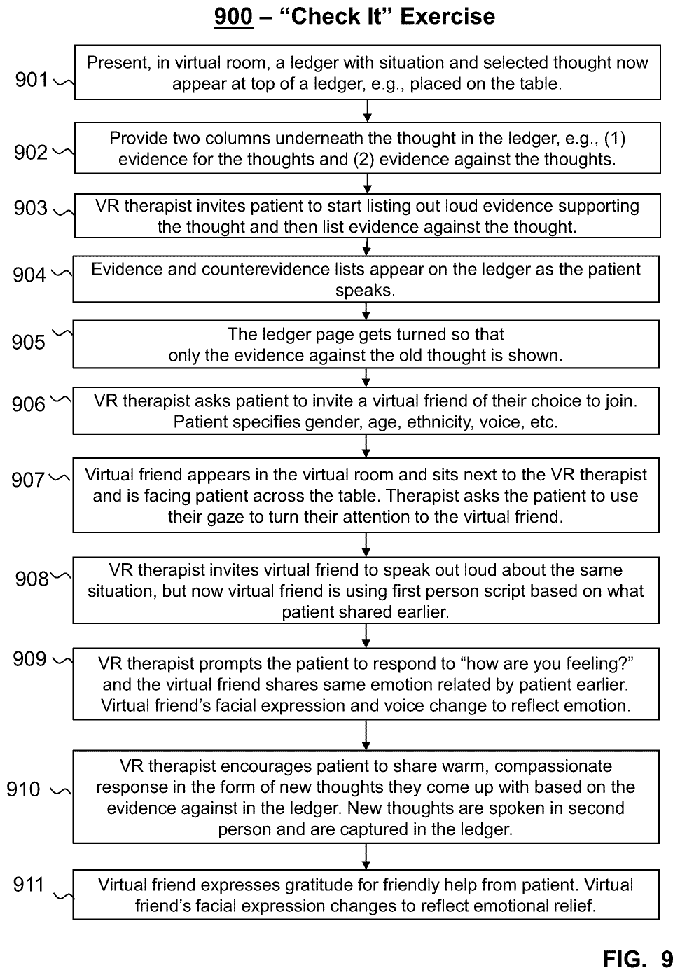

To continue with Cognitive Therapy according to this disclosure, FIG. 9 illustrates the “Check It” exercises, e.g., in process 900. For the “Check It” exercises, the therapist invites the patient back to a virtual therapy room, in Step 901. Still in Step 901, present, in virtual room, a virtual ledger (or other type of book or paper) with a situation and a selected thought may now appear at the top of a ledger, e.g., placed on the table.

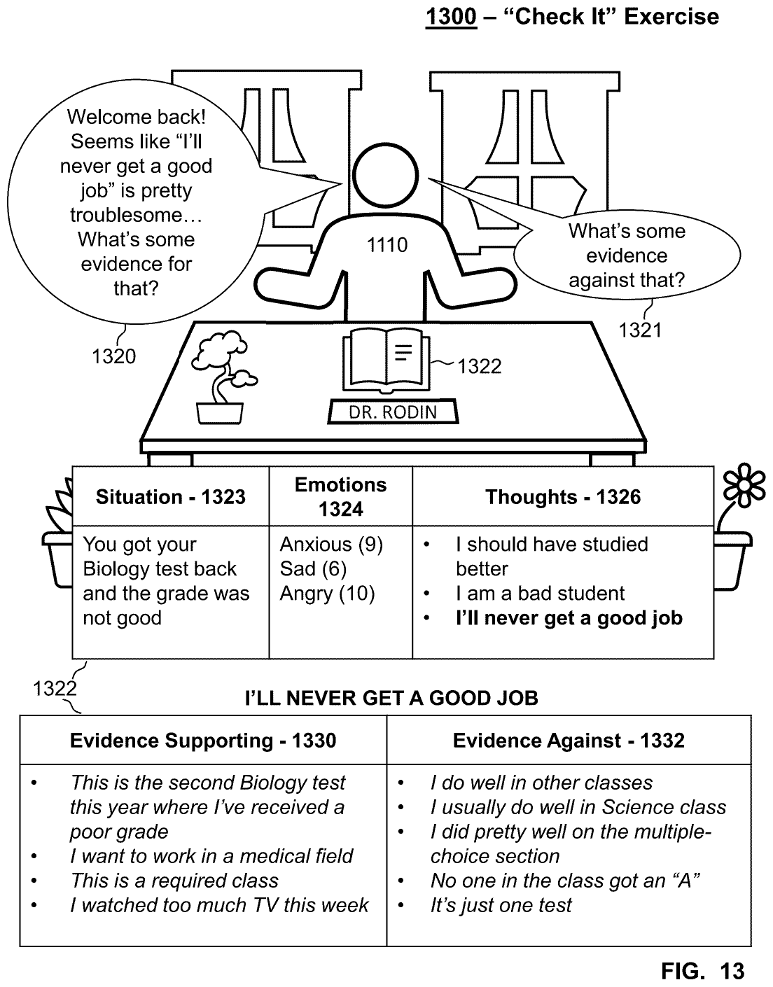

In Step 902, as illustrated in this example, the VR therapist provides two columns underneath the thought in the ledger, e.g., (1) evidence for the thoughts in a first column and (2) evidence against the thoughts in a second column. Some parts of these steps may be depicted as portions of FIG. 13. In Step 903, the VR therapist invites the patient to start listing out loud evidence supporting the thought and then list evidence against the thought. In Step 904, the VR therapist provides the evidence and counterevidence as lists that appear on the ledger as the patient speaks. In Step 905, the ledger page gets turned on the ledger so that only evidence against the old thought is shown. In Step 906, the VR therapist asks the patient to invite a virtual friend of their choice to join. Similarly, the user or patient may customize the characters of the virtual friend. The patient may specify the gender, age, height, weight, body style, ethnicity, voice, hair style, clothing style, etc. of the virtual friend.

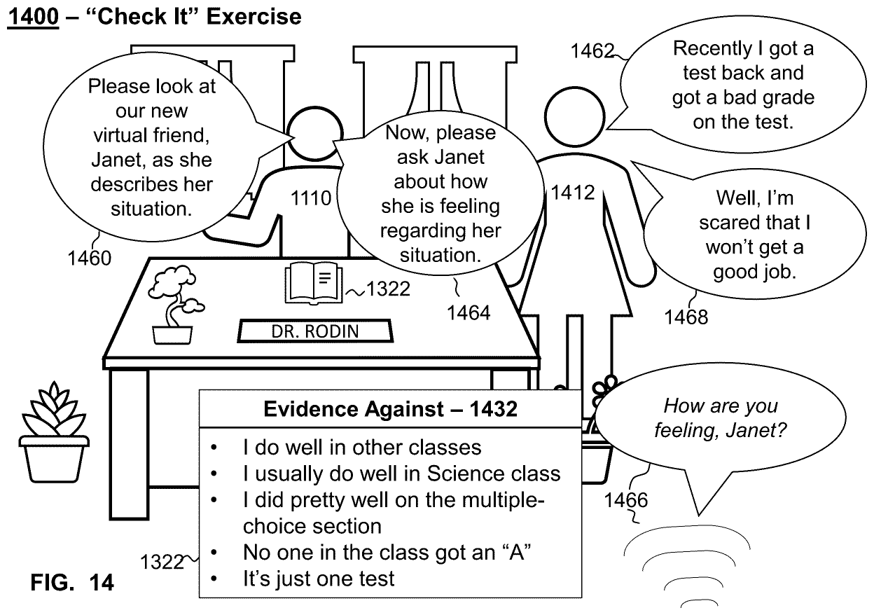

In Step 907, in one embodiment, the virtual friend appears in the virtual room and sits next to the VR therapist and is facing patient across the table. The therapist avatar may ask the patient to use their gaze to turn their attention to the virtual friend. In Step 908, the VR therapist then invites the virtual friend to speak out loud about the same situation, but now the virtual friend uses a first-person script based on what patient shared earlier. In Step 909, the VR therapist prompts the patient to respond, saying, “How are you feeling?” and virtual friend shares the same emotion related by the patient earlier. The virtual friend's facial expression and voice may change to reflect an emotion. Some parts of these steps may be depicted as portions of FIG. 14. In Step 910, the VR therapist encourages the patient to share a warm, compassionate response in the form of new a thought the patient may think of based on the evidence against from the ledger. Such new thoughts should be spoken in second person and are captured in the ledger. In Step 911, the virtual friend expresses gratitude for friendly help from the patient. The virtual friend's facial expression may change to reflect emotional relief.

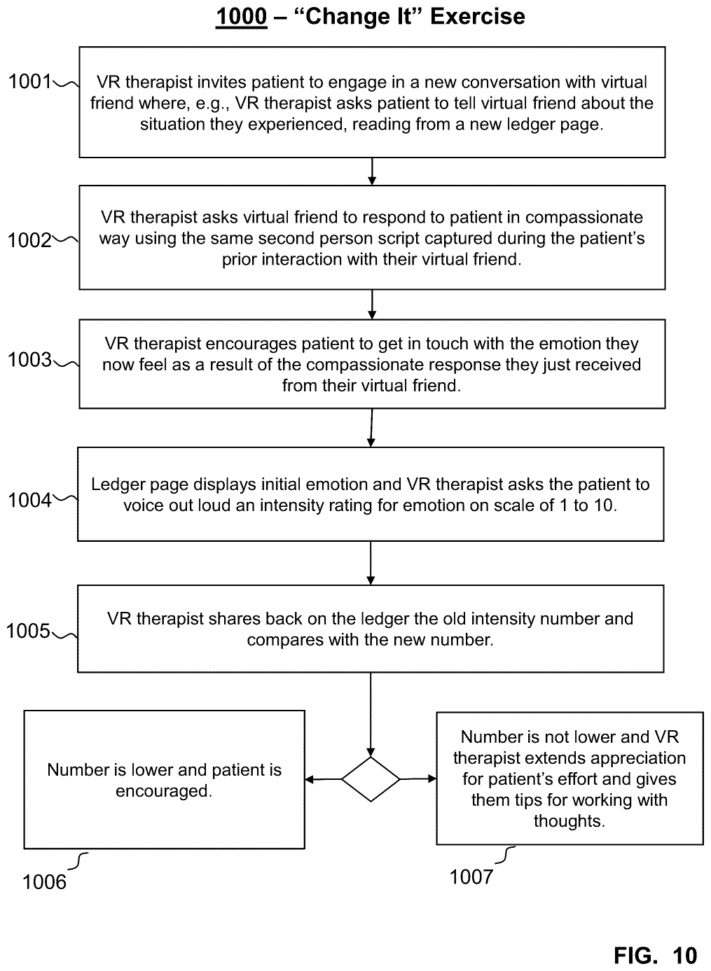

To complete the Cognitive Therapy according to this disclosure, FIG. 10 illustrates the “Change It” exercises. At Step 1001, the VR therapist invites the patient to engage in a new conversation with the virtual friend. This time, the VR therapist asks the patient to tell the virtual friend about the situation they experienced, reading from a new ledger page. At Step 1002, the VR therapist then asks the virtual friend to respond to the patient in a compassionate way using the same (or similar) second-person script captured during the patient's during their prior interaction with their virtual friend. At Step 1003, the VR therapist encourages patient to get in touch with the emotion they now feel as a result of the compassionate response they just received from their virtual friend.

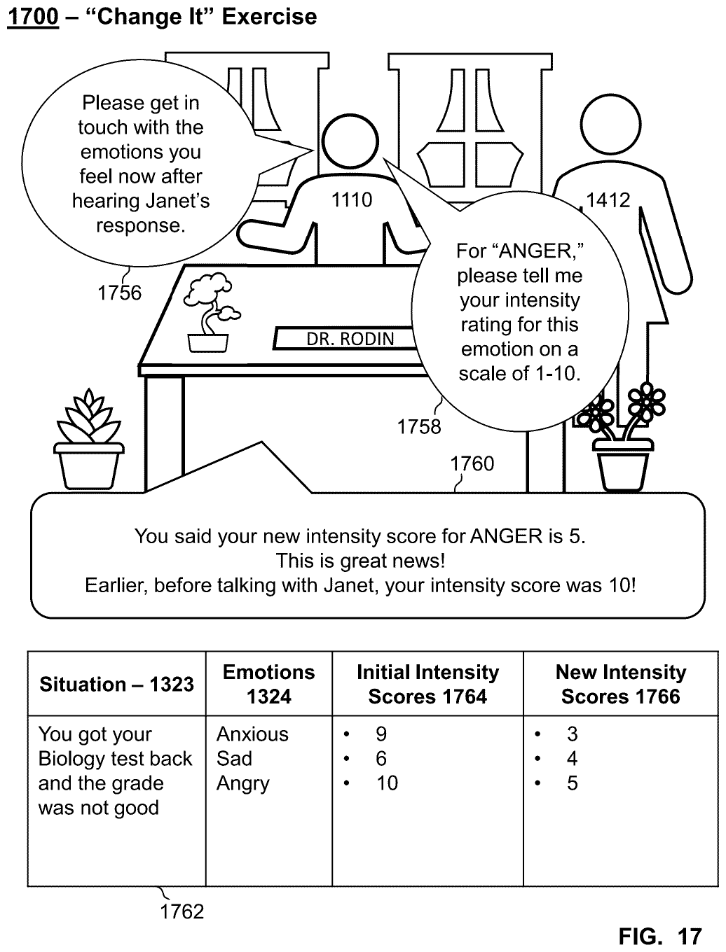

At Step 1004, the ledger displays the initial emotion, e.g., appearing on a new ledger page. The therapist asks the patient to voice out loud an intensity rating for emotion, e.g., on a scale of 1 to 10. Some parts of these steps may be depicted as portions of FIG. 17. At Step 1005, the VR therapist shares back, e.g., via the ledger, the original intensity number and compares with the new intensity rating. The new intensity rating number should be lower, and, at step 1006, the patient is given encouragement and/or congratulations. If the new intensity rating number is not lower, then at Step 1007, the VR therapist extends appreciation for patient's effort during the exercises and may give the patient tips for working with thoughts.

FIG. 11 is an illustrative depiction of a user interface, e.g., a virtual therapist's office, for a VR therapy platform, in accordance with some embodiments of the disclosure. Scenario 1100 of FIG. 11 depicts, e.g., a VR avatar entering a virtual office or a virtual therapist 1110. The therapist office depicted in scenario 1100 is exemplary of a potential setting that may encourage a patient to feel comfortable and forthcoming in his or her feelings and thoughts. In scenario 1100, virtual therapist 1110 may initiate a discussion about a patient's current thoughts, feelings, emotions, and recent situations via audio and/or visual cues. This may be considered a beginning portion of the “Catch It” exercises.

Scenario 1100 may be displayed to a patient view via the head-mounted display, e.g., “Patient View.” In some embodiments, a head-mounted display (HMD) may generate a Patient View as a stereoscopic 3D image representing a first-person view of the virtual interface with which the patient may interact. An HMD may transmit Patient View, or a non-stereoscopic version, as “Spectator View” to, e.g., a clinician tablet for display.

Prior to entering a VR environment, a patient may choose characteristics of their avatar such as height, weight, skin color, gender, clothing, etc. In some embodiments, a patient may also choose characteristics for a therapist avatar such as height, weight, skin color, gender, hairstyle, clothing style, etc. Avatar customization may be important, in some embodiments, e.g., in order to help make the patient more comfortable with talking and more susceptible to correcting assumptions and/or “thinking errors.” Avatar customization may be a straightforward user interface or series of menus. In some embodiments, a patient profile may be recorded and the avatar customization(s) associated with the patient and/or device may only need to be entered once. The avatar customizations may be stored in a patient or therapist profile, e.g., in local memory and/or at in a cloud server. Once physical and/or visual parameters for one or more avatars are input, or accessed from saved preferences, avatars may be rendered based on the parameters using VR application based on, e.g., software-development environment.

In scenario 1100, a patient avatar may enter a virtual room or setting such as a virtual therapy room. Once the patient avatar is in the virtual room, the patient may acclimate herself to the virtual world. For instance, a patient may view the hands of their avatar in front of their face or resting on their lap. To facilitate comfortability in the virtual environment, a patient may be asked to raise their hands in front of headset and move them. Some embodiments may use electromagnetic trackers, e.g., as depicted in FIGS. 18-21.

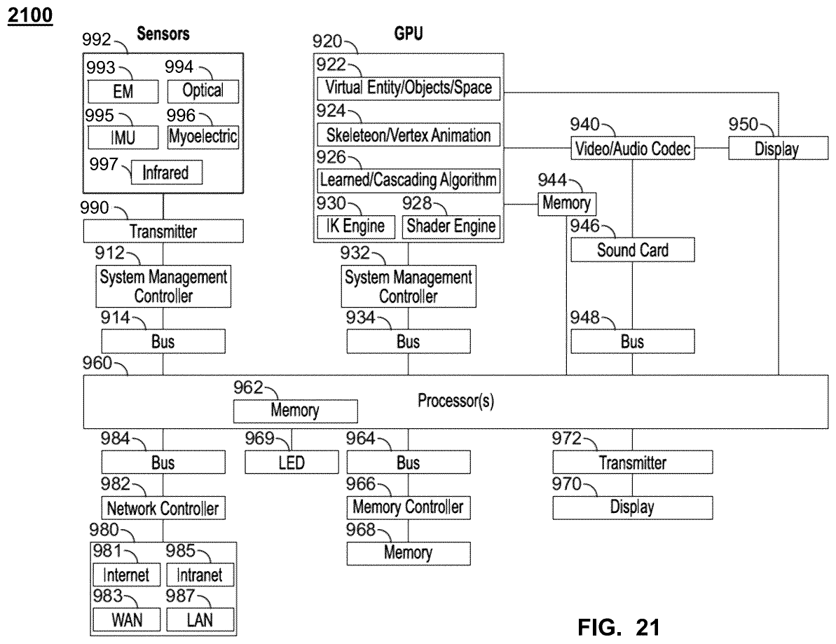

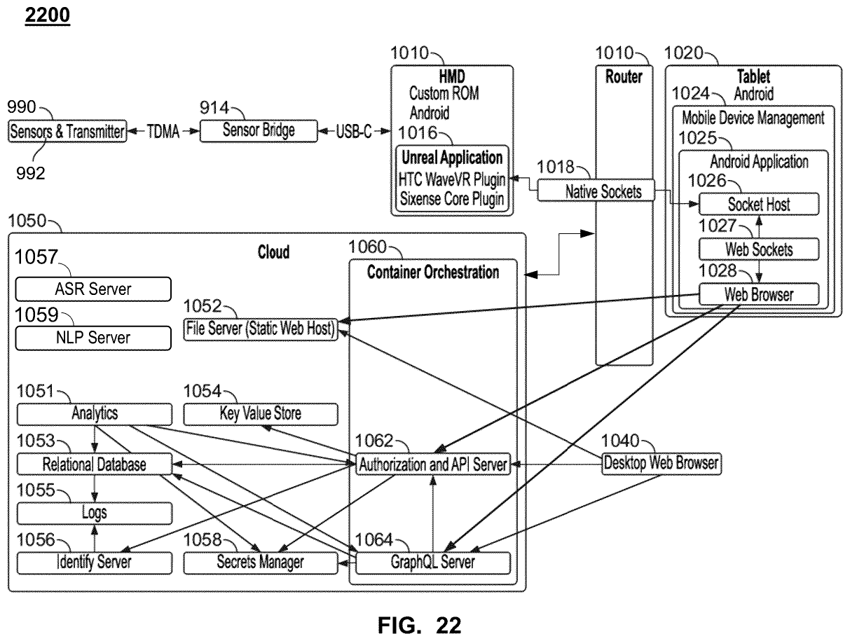

In scenario 1100, virtual therapist 1110 may initiate a discussion about a patient's current thoughts, feelings, emotions, and one or more recent situations via audio and/or visual cues. In some embodiments, non-playable characters depicted in the virtual world, such as virtual therapist 1110, may speak or provide thoughts via one or more audio and visual interactions. For instance, virtual therapist 1110 may provide animated speech and audio prompts, questions, comments, requests, responses, summarizations, suggestions, etc. In some embodiments, the VRCT platform may provide subtitles and/or captions. In scenario 1100, and other scenarios throughout this disclosure, speech balloons such as prompt 1120 and/or response 1124 may depict the substance of provided audio. Audio provided by the VRCT platform may comprise instruction and/or conversation with virtual characters, e.g., via text-to-speech services. An HMD may provide audio via a sound card, e.g., sound card 946 of FIG. 21. FIG. 22 also depicts speech and voice services. In some embodiments, speech may be provided as visual depictions of a conversation. For instance, in circumstances where audio is muted and/or a patient is hearing impaired, text may be provided, e.g., as speech balloons, captions, and/or subtitles.

In scenario 1100, or just prior to, a virtual therapist avatar 1110 may enter the virtual room and take a seat across from the patient avatar. As depicted in scenario 1100, there may be a desk or table between the avatars of the therapist and the patient, along with other virtual objects that may be considered as potentially making a patient feel more relaxed or comfortable. In some embodiments, therapist avatar 1110 may be designed to make eye contact and/or mimic poses of one or more patient body parts (with some randomness and/or delay), e.g., to seem more likeable and approachable. In scenario 1100, the patient may be invited to use her gaze to start engaging with the therapist avatar. In some embodiments, gaze may be approximated by determining head position via sensors on the HMD (see FIGS. 7-10). In some embodiments, eye tracking technology may be used in the HMD. For instance, Tobii is a supplier that uses camera-based eye tracking, and Adhawk supplies a MEMS-based eye tracking technology.

In scenario 1100, or just prior to, a virtual therapist avatar 1110 welcomes the patient and proposes to start a Cognitive Therapy session related to a situation that is triggering negative emotions. For instance, virtual therapist avatar 1110 may offer prompt 1120, saying, “Please tell me about the recent situation that was triggering negative emotions . . . ” Some embodiments may use, e.g., conversational text generation. For instance, the text that the therapist avatar will speak in/around scenario 1100 could be scripted. In some embodiments, appropriate text for the situation can be generated using a neural network, such as OpenAI®'s GPT-3. Speech synthesis and text-to-speech services may be used to take textual data and convert it to synthesized spoken audio. In some embodiments, therapist avatar 1110 may be animated to visually appear to speak the words, e.g., of prompt 1120. In some embodiments, speech animation and/or avatar lip sync may be configured using several commercially available systems, including Speech Graphics and JALI Research. Some embodiments may provide text transcripts of dialogue from a virtual therapist.

In scenario 1100, after therapist avatar 1110 invites the patient to describe a situation, a patient may respond. In some embodiments, the VRCT platform will receive voice input 1122, e.g., using a microphone in connection with the HMD (e.g., via sound card or USB interface). For instance, patient voice input may be captured as an audio signal using the microphone built into the HMD. In some embodiments, ASR and NLP may be used in receiving the voice input. Some embodiments may use a third-party speech-to-text service where, e.g., an audio signal is converted into text using speech recognition tools in the cloud. For example, Amazon® and Microsoft® each have speech-to-text transcription cloud services.

In scenario 1100, therapist avatar 1110 provides response 1124 to, e.g., reflect what was captured and comprehended from voice input 1122. In some embodiments, a response such as response 1124 may request confirmation. Response 1124, for instance, says, “So, what I'm hearing is that yesterday you got your Biology test back and the grade was not good even though you studied for it . . . . Is that correct?” At this point in scenario 1100, the patient can either confirm or reject the response. In some embodiments, the patient may provide confirmation via voice, gaze, and/or other input. In some embodiments, the patient may provide additional voice input, like voice input 1122, to restate information about the described situation.

In some embodiments, reflecting the captured situation information of the patient, text may be processed using a neural-network-based auto-summarization (e.g., an “auto-summarizer”). For example, OpenAI®'s GPT-3 supports auto-summarization where, e.g., a desired length of the summary may be input as a parameter and a summary generated. If the patient accepts the summary, the interaction continues. In some embodiments, if the patient rejects the summary and specifies a clarification, a new summary may be generated. In some implementations, if no further clarification is provided by the patient, the virtual therapist (or the VRCT platform) may generate a new summary of the original situation with a different length (e.g., 25-33% longer or shorter).

In some embodiments, the patient may elaborate about the situation, e.g., using a voice input, and an auto-summarizer may be applied solely to the elaboration. In some embodiments, the auto-summarizer may be applied to the original explanation combined with any elaboration or supplements provided.

In some embodiments, upon confirmation of a correct capture and comprehension by the virtual therapist and/or VRCT platform, scenario 1100 may progress to a next scenario, such as scenario 1200 as depicted in FIG. 12. For instance, after a patient confirms, the VRCT platform therapist may take the patient to a setting such as a virtual nature environment with lake 1214, e.g., for a guided mindfulness inquiry. In some embodiments, this may be a prerecorded guided practice. The lake nature environment may be a familiar VR setting for mindfulness exercises. Dialogue processed in the virtual journey to the mindfulness setting may be processed similarly as with other dialogue between the virtual therapist and the patient. In some instances, virtual therapist 1210 of scenario 1200 may have a different costume or different appearance from therapist avatar 1110 of scenario 1100 in FIG. 11.

During the mindfulness inquiries portion of the “Catch It” exercises, depicted as scenario 1200 of FIG. 12, the virtual therapist avatar 1210 may invite the patient to think about the situation that is linked to their negative emotions. For instance, in prompt 1211, the virtual therapist says “Which are the predominant emotions you felt regarding this situation? Use your gaze to select them.” In some embodiments, scripted audio may be prerecorded and played, or text-to-speech services as described above. In some embodiments a speech balloon or caption may be generated with or instead of audio. The virtual therapist may ask the patient to use their gaze (or another input) to select one or more predominant emotions from different emotions that are bubbling up at the surface of lake 1214. For instance, emotions 1222-1258 as depicted in scenario 1200 of FIG. 12 may appear to bubble from lake 1214. Such emotions may include, e.g., cautious 1222, happy 1228, sad 1226, shy 1230, frustrated 1232, empty 1240, embarrassed 1242, angry 1244, worried 1246, anxious 1248, overwhelmed 1254, hopeful 1256, guilty 1236, nervous 1234, shocked 1252, and more. To select an emotion with gaze, in some embodiments, a cursor may be moved based upon gaze (e.g., HMD movement and/or eye tracking) and a bubble may be selected by holding the gaze until a time tracker fills up. For instance, time tracker 1245 depicts, e.g., 4 seconds of a 6-second time tracker have filled up based on a held gaze for the angry 1244 bubble. In some embodiments, the patient may select one or more emotions using speech, gaze, and/or by pointing at the emotion bubble icon with their hands. In some embodiments, a patient may be asked to speak selected emotions aloud for audio capture. Scenario 1200 depicts that multiple bubbles may be selected; however, some embodiments may only use one selected bubble. Bubbles may be considered representative icons or shapes used for emotions, and other icons or shapes may be substituted; however the extended analogy of emotions “bubbling up” in scenario 1200 may prove to make patients more relaxed and/or engaged.

In some embodiments, a prompt from the virtual therapist may be triggered by the detection of a strong emotion from physiological sensors (e.g., during a “Catch It” exercise). For instance, if a heart rate monitor measures heart rate above a threshold (e.g., 150 beats per minute), bubbles similar to, e.g., anger, stress, anxiety, etc. may be brought to the forefront or top or made to be larger than other surrounding bubbles. In some embodiments, ordering and placing of the emotion bubbles may be based on the likelihood of detected emotions by physiological measures.

In some embodiments, further therapist dialogue (or moving to the next step) may be triggered by a timeout. For instance, after a 40-second countdown and/or 10-15 seconds of inactivity, the virtual therapist may ask the patient to confirm the emotions and/or ask if the patient is ready to move on.

In some embodiments, the virtual therapist may ask the patient to speak the intensity level of their emotion, e.g., on a scale of 1 to 10. For instance, a virtual therapist may say, “On a scale of 1 to 10, what is the intensity level you feel for the selected emotion?” A rating meter may allow a gaze-based input using icons and/or colors/shades to reflect the available values on a scale. In some embodiments, an intensity value may be input by voice or other input. In some embodiments, the color intensity of each emotion bubble reflects the emotion intensity level, e.g., bright red for intense anger (angry bubble 1244). In some cases, bubble size may reflect intensity. In some embodiments, a default selection of intensity level may be set according to a predicted intensity based on physiological signals. For example, if a connected heart rate monitor measures a high heart rate, (e.g., over 1200 beats per minute), a predicted intensity at the top of the scale may be used for the patient. In some embodiments, an intensity level may be recorded for each selected emotion, e.g., sad 1226, anxious 1258, and angry 1244.

In some embodiments, the bubbles of lake 1214 may be removed to make way for a new icon or shape, e.g., clouds, to rise as thoughts are spoken by the patient. For instance, therapist avatar 1210 may invite the patient to allow her mind to wander into thoughts related to the situation and speak them as they arise, saying in a prompt, e.g., “Let your mind wander into thoughts related to the situation and speak them as they come to mind . . . the thoughts you speak will arise from the lake.” In some embodiments, spoken thoughts may appear on virtual cloud icons. In some embodiments, a speech-to-text service may be used again to convert spoken audio input to text. Some embodiments may use natural language processing, e.g., machine learning. For instance, some thoughts for such a situation may be “I should have studied better,” “I'll never get a good job,” “Biology is my worst subject,” “It was an important test,” “I should just quit school,” and “I am a bad student.” In some embodiments, the virtual therapist may help weed out thoughts that are not workable, e.g., thoughts that are an expression about emotions. For example, a patient may say, “Being sad is awful” or “I hate school.” Some embodiments may use keywords to filter out emotional phrases. Some embodiments may use NLP to identify and filter such statements. In some embodiments, the virtual therapist may ask the patient to select a most troublesome thought. For instance, a prompt may request the patient select a thought that is the most troublesome or concerning with her gaze. In some embodiments, once selected, only that thought remains in a cloud and the rest disappear.

In some embodiments, after the mindfulness exercise(s) at the lake, the virtual therapist may politely invite the patient to come back to the virtual therapy room before the setting is changed.

Once the patient and virtual therapist 1110 are back in the virtual therapy room, as depicted in scenario 1300 of FIG. 13, the “Check It” exercises may begin. In scenario 1300, the VRCT platform displays ledger 1322 and prompts the patient to voice evidence supporting the selected thought and evidence refuting the selected thought. For instance, virtual therapist avatar 1110 may say, “Welcome back! Seems like ‘I'll never get a good job’ is pretty troublesome . . . . What's some evidence for that?” as depicted in prompt 1320. In scenario 1300, the description of the situation 1323, emotions 1324, and selected thoughts 1326 may now appear on top of a page in ledger 1322, e.g., placed on the table between the therapist and the patient. For instance, situation 1323 may say “You got your Biology test back and the grade was not good”; emotions 1324 may say “Anxious (9), Sad (6), Angry (10)” representing emotions and intensity values; and selected thoughts 1326 may include, e.g., “I should have studied better,” “I am a bad student”; and the selected thought: “I'll never get a good job.” In some embodiments, the ledger may be oriented so that the patient may read it. In some embodiments, the ledger may be stored as a data structure such as a table, matrix, database, linked list, etc.

In scenario 1300, two columns appear underneath the selected thought (e.g., “I'll never get a good job”): evidence supporting the thought 1330 and evidence against the thought 1332, e.g., as part of ledger 1322. With prompt 1320, the virtual therapist invites the patient to start listing out loud evidence for the thought one piece of evidence at a time. Then the virtual therapist invites the patient to start listing out loud evidence against the thought one piece of evidence at a time. Ledger 1322 is filled with patient statements with evidence supporting 1330 and evidence against 1332, e.g., as captured by audio and converted to text (e.g., ASR/NLP).

In some embodiments, evidence supporting 1330 and evidence against 1332 may be filled separately, one at a time, e.g., with evidence supporting 1330 and evidence against 1332 second. In some embodiments, evidence supporting 1330 first and evidence against 1332 may be filled at the same time with the patient identifying each statement as evidence supporting or evidence against. For example, such identification may be made with speech, or, in some cases, the VRCT platform may use eye tracking or gaze tracking to specify the focus of the input to either column. When capturing evidence for and against is complete, the VRCT platform may proceed from scenario 1300 to scenario 1400 of FIG. 14.

As part of the “Check It” exercise, scenario 1400 of FIG. 14 depicts, e.g., that a page is turned in ledger 1322 and the virtual therapist brings a friend avatar to talk through a similar situation. With regard to ledger 1322 in scenario 1300, only evidence against the (initial) thought is depicted. In scenario 1400, virtual friend 1412 appears in the virtual room and, e.g., stands or takes a seat next to the therapist avatar 1110, e.g., facing the patient across the table. Therapist avatar 1110 asks the patient to turn their attention to (e.g., using their gaze) and greet a virtual friend 1412 in prompt 1460, saying, “Please look at our new virtual friend, Janet, as she describes her situation.” In some embodiments, virtual friend 1412 is an avatar designed by the patient, e.g., using her choice of gender, age, ethnicity, etc., so as to feel most comfortable in the experience. In some embodiments, customizing a friend avatar may use the same (or similar) interface as customizing a patient or therapist avatar. Customizing avatars based on input and/or preferences may help a patient feel more comfortable with CT therapy. In some embodiments, avatar generation for a virtual friend may be generated from a photo of a real friend using a third-party service such as Itseez3D AvatarSDK, Spatial, or Ready Player Me.

Further in scenario 1400, e.g., when the patient turns her gaze to the virtual friend, virtual friend 1412 relays information, in statement 1462, about a situation that is very similar to the situation provided by the patient. For instance, virtual friend 1412 may speak out loud to the patient about the same situation shared by the patient earlier, but now from the perspective of the virtual friend going through the experience. Statement 1462 of scenario 1400 comprises: “Recently I got a test back and got a bad grade on the test,” while situation 1323 from ledger 1322 in FIG. 13 comprises: “You got your Biology test back and the grade was not good.” Altering the perspective or point of view of a statement may be performed using, e.g., NLP, word replacement, and/or syntax/grammar correction. Changing the statement structure from second-person to first-person, so that the patient may hear a virtual friend saying a similar statement from his/her own perspective, may be a valuable part of the VRCT's “Check It” exercises.

In some embodiments, a virtual friend may be using a synthetized voice. For instance, a virtual friend may be using a first-person script based on what patient shared earlier about the situation using NLP and text-to-speech services. Some embodiments may use, e.g., voice cloning and/or voice conversion to allow a virtual avatar to speak with the voice of a patient's real friend with services such as Descript's Overdub and Respeecher. To script the virtual friend's speech, the VRCT platform may use speech synthesis directly with a model of a selected real-world friend's voice to create spoken audio, e.g., for scenario 1400. In some embodiments, the VRCT platform may generate speech in any voice and then use voice conversion to modify the speech to the selected voice of the virtual friend.

At prompt 1464 of scenario 1400, the virtual therapist encourages the patient to respond to statement 1462 from virtual friend 1412 with a question, e.g., “Now, please ask Janet about how she is feeling regarding her situation.” The VRCT platform receives patient-provided audio input 1466: “How are you feeling, Janet?” In response, virtual friend 1412 may share the same emotion and/or thoughts provided by the patient earlier. For instance, in response 1468, virtual friend 1412 states: “Well, I'm scared that I won't get a good job,” which is similar to thoughts 1326 stored in ledger 1322.

In some embodiments, the virtual friend's facial expression and voice may change to reflect the emotion, e.g., using emotion matching. In some embodiments, avatar facial expression rendering may use, e.g., Facial Action Coding System (FACS)-based avatar rigs to characterize facial behaviors based on facial musculature. Many avatar-generating systems now support FACS-based rigs, so that the avatar may be easily morphed using FACS controls. Certain facial expressions may be commonly associated with specific emotions, and may be characterized as a collection of facial action units.

When an avatar is rigged using FACS controls, the specific action units are exposed as parameterized controls that may be manipulated directly. The VRCT platform may control the intensity of such variables as, e.g., “Cheek Raiser” and “Lip Corner Puller” directly to animate emotions. Some embodiments may use emotion-based avatar rigs.

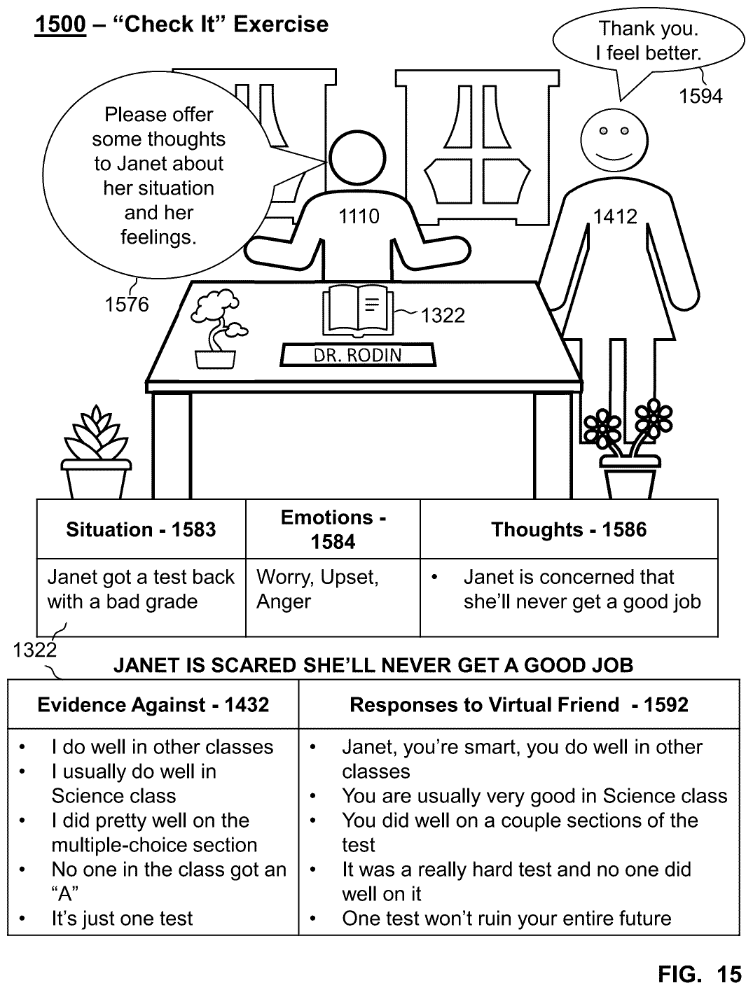

As part of the “Check It” exercises, in scenario 1500, depicted in FIG. 15, therapist avatar 1110 encourages the patient to share a warm, compassionate response based on new thoughts they came come up with developed from the recorded evidence against the (initial) thought, e.g., as shown in ledger 1322. For instance, a turned page in ledger 1322 depicts situation 1583, “Janet got a test back with a bad grade,” emotions 1584, “Worry, Upset, Anger,” and thoughts 1586 “Janet is concerned that she'll never get a good job” for virtual friend 1412. Of course, the virtual friend's situation, emotions, and thoughts are based on the patient's recorded situation, emotions, and thoughts.

In scenario 1500, the virtual therapist, in prompt 1576, asks the patient: “Please offer some thoughts to Janet about her situation and her feelings.” New thoughts may be spoken in a second-person perspective and captured on the ledger next to (or on top of) the list of evidence against such thoughts. For instance, in response to virtual friend 1592, a patient might say: “Janet, you're smart, you do well in other classes,” “You are usually very good in Science class,” “You did well on a couple sections of the test,” It was a really hard test and no one did well on it,” and “One test won't ruin your entire future” based on evidence against 1432 saying, e.g., “I do well in other classes,” “I usually do well in Science class,” “I did pretty well on the multiple-choice section,” “No one in the class got an ‘A,’” and “It's just one test.” Capture of these statements may be performed with ASR/NLP. In some embodiments, the patient may be prompted to read the evidence against 1432 and speak in second-person statements to virtual friend 1412. In some embodiments, the patient may be provided one or more examples of responses to virtual friend 1592 as based on evidence against 1432 and encouraged to read and speak in second-person statements to virtual friend 1412. Such examples may be provided by using a grammar shift, e.g., from first-person statements to second-person statements, using NLP. Each statement of responses to virtual friend 1592 may be captured and separated based on pauses and or further NLP. The patient may affirm she is finished, or there may be a timeout after, e.g., 45 seconds.

In some embodiments, the virtual friend may express gratitude for the friendly and/or empathetic responses to virtual friend 1592 by saying a statement 1594 and/or changing facial expressions to reflect emotional relief. In some embodiments, virtual friend 1412's expression may be reflected non-verbally using a FACS-based avatar rig and/or verbally using emotional speech synthesis and speech-based avatar expression rendering, as described above.

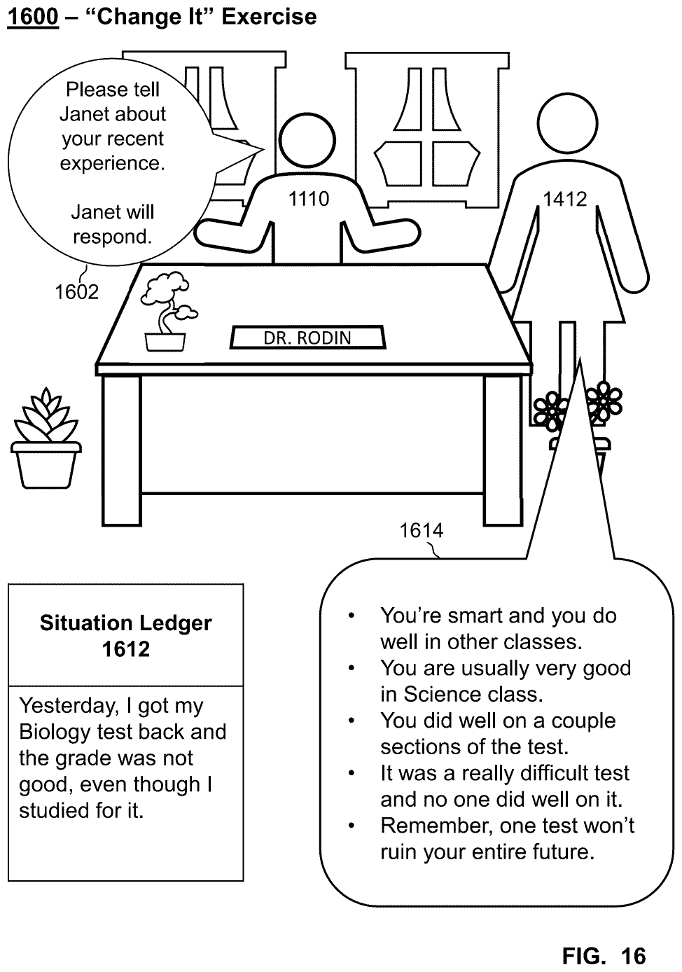

In scenario 1600, as depicted in FIG. 16, virtual therapist 1110 invites the patient to engage in a new conversation with virtual friend 1412. Scenario 1600 may be considered part of the “Change It” exercises. Here, virtual therapist 1110 may ask the patient to tell virtual friend 1412 about the situation she experienced. Prompt 1602 states, “Please tell Janet about your recent experience . . . . Janet will respond.” The patient may be encouraged to read from a newly generated ledger page, situation ledger 1612, based on the original situation relayed by the patient. Situation ledger 1612 includes the statement “Yesterday, I got my Biology test back and the grade was not good, even though I studied for it.”

The situation of situation ledger 1612 may be retrieved from the earlier conversation (situation 1323 of ledger 1322) and displayed for the patient to read. Then virtual therapist 1110 may ask virtual friend 1412 to respond to the patient, e.g., in a compassionate way, using the same second-person script used during the patient's prior interaction with their virtual friend, responses to virtual friend 1592. Virtual friend 1412's compassionate response 1614 may include, e.g., “You're smart, you do well in other classes,” “You are usually very good in Science class,” “You did well on a couple sections of the test,” It was a really difficult test and no one did well on it,” and “Remember, one test won't ruin your entire future” Again, some embodiments, may use text-to-speech, NLP, and/or ASR services to generate response 1614.

After response 1614, advancing to scenario 1700 depicted in FIG. 17, virtual therapist 1110 may encourage the patient to get in touch with the emotion she now feels as a result of the compassionate response 1614 she just received from virtual friend 1412. Prompt 1756 of scenario 1700 says, e.g., “Please get in touch with the emotions you feel now after hearing Janet's response.” In scenario 1700, ledger page 1762 is provided with, e.g., situation 1323, “You got your Biology test back and the grade was not good,” emotions 1324, “Anxious, Sad, Angry,” initial intensity scores 1764, “9, 6, 10,” respectively. Virtual therapist 1110 asks the patient to voice out loud an intensity rating for each of emotions 1324, e.g., on a scale of 1 to 10. For instance, in prompt 1758, therapist avatar 1110 asks, “For ‘ANGER,’ please tell me your intensity rating for this emotion on a scale of 1 to 10.” Such ratings may be input with voice and/or other input and/or selection methods. In response to receiving an input of new intensity scores 1666, new intensity scores 1766, “3, 4, 5,” respectively, are displayed next to initial intensity scores 1764 on ledger page 1762.

In scenario 1700, after receiving new intensity scores 1766, the VRCT platform compares the initial intensity scores 1764 with new intensity scores 1766. Generally, the new intensity scores 1766 should be lower. In the event that new intensity scores 1666 are not lower than initial intensity scores 1764, the news may be shared and the encouragement and congratulations may be offered. For instance, response 1760 states, “You said your new intensity score for ANGER is 5. This is great news! Earlier, before talking with Janet, your intensity score was 10!”

In some embodiments, in the event that new intensity scores 1766 are not lower than initial intensity scores 1764, virtual therapist 1110 extends appreciation for the patient's effort, and may provide tips for working with, e.g., thought errors and specific thoughts. In some embodiments, the process may start over. In some embodiments, the process may rewind to a prior stage, e.g., the lake. In some embodiments, some meditation and/or other mindfulness exercises may be provided.

In some embodiments, patient-reported emotions and/or values may not be the only input. Biometric data, such as data measured by biometric sensors like the devices depicted in FIG. 7, may be taken at various points during VR Cognitive Therapy. For instance, a patient's heart rate and/or blood pressure may be measured at a predetermined interval and/or at certain points of therapy to track whether a patient's emotional state is improving. In some embodiments, a biometric value may be recorded at the beginning of and/or end of, e.g., each of the “Catch It,” “Check It,” and “Change It” exercises. For example, a heart rate baseline may be set at the beginning of therapy and monitored at intervals for comparison to determine whether each exercise is helping (or exacerbating) the patient's heart rate. Similarly, perspiration sensors may be used to set an initial value and monitor whether each exercise results in an increase or decrease in perspiration. In some embodiments, image sensors used to, e.g., track facial expressions, eye movement, and/or facial reflexes may record initial values for comparison at different intervals and/or during portions of each Cognitive Therapy exercise. In some embodiments, biometric data may be used to supplement and/or adjust patient-reported data. For instance, in VRCT, a patient may be down-playing or exaggerating an intensity level of an emotion or thought. Cognitive Therapy typically works best when a patient is honest, but patients may not always be genuine and/or open to therapeutic assistance. Additional data, may be used for comparison to patient-reported data to identify discrepancies and/or need for reconciliation. Some discrepancies may lead to adjustment of patient feedback data while some may be weighted or reconciled based on other patient data such as underlying conditions. Patient biometric data may be taken before, during, or at the end of a VRCT exercise and used as a comparison. For instance, an initial intensity level for anger may be lowered based on a low(er) reading for a heart rate or perspiration level. In some cases, charts may be developed for therapists and doctors to observe discrepancies over time.

In some embodiments, biometric data may be used to supplement and/or adjust patient-reported data. For instance, in some embodiments, biometric values may be used in conjunction with patient input about emotional state and/or intensity values. In some embodiments, biometric data may be used to supplement and/or compare to patient survey data. For instance, a patient may take a survey, such as the PHQ-9 (Patient Health Questionnaire-9), a multipurpose instrument for screening, diagnosing, monitoring, and measuring the severity of depression and biometric data may be normalized and compared to responses and/or scores. In some embodiments, neural networks may be trained based on survey data and biometric data and used to determine if new biometric data may indicate a patient might relapse, staying steady, or improving. In some cases, surveys such as the PHQ-9 may validate whether a patient's emotional state is improving, e.g., as indicated by biometrics and other feedback.

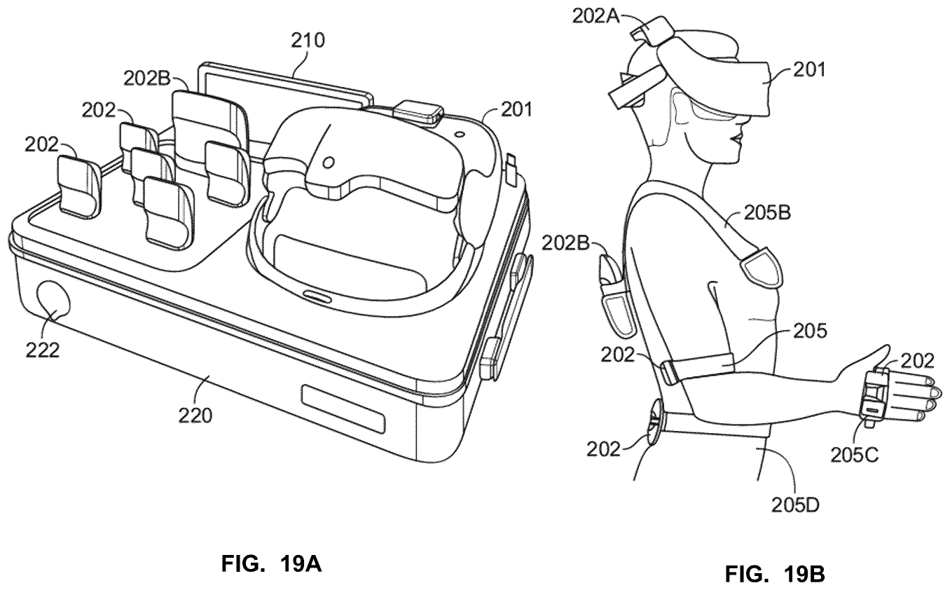

FIG. 18A illustrates a flow-chart for an exemplary process for collecting biometric feedback, in accordance with some embodiments of the present disclosure. There are many ways to use biometrics, e.g., along with patient response/input, for treating a patient, and process 1800 is one example. Some embodiments may utilize a VRCT engine to perform one or more parts of process 1850, e.g., as part of a VR application, stored and executed by one or more of the processors and memory of a headset, server, tablet and/or other device. For instance, a VRCT engine may be incorporated in, e.g., as one or more components of, head-mounted display 201 and/or other systems of FIGS. 19-22.

Generally, a VRCT engine may receive and record a biometric value at the beginning of a therapy session, at the end of therapy session, and/or during each of a plurality of exercises, e.g., the “Catch It,” “Check It,” and “Change It” exercises. FIG. 18B is an illustrative chart, chart 1820, depicting collected biometric data, in accordance with some embodiments of the present disclosure. FIG. 18B depicts an exemplary chart based on an illustrative biometric measurement, e.g., beats per minute for a heart rate, recorded over time. The numbered steps in chart 1820 of FIG. 18B correspond to the steps of process 1800 of FIG. 18A. With a successful therapy session, a chart like FIG. 18B will track biometric data depicting a patient's emotional state improving, e.g., experiencing less intensity for one or more emotions and/or thoughts over the session or calming down. If biometric data indicates more intense emotions, e.g., above a threshold like 20% higher, another therapy may be needed. In some embodiments, therapy exercises may affect a patient and her biometric data differently, but the end goal of VRCT will be to achieve a measurement indicating that the exercises together will improve the emotional state of the patient. For instance, a decline in a biometric data indicating more calmness—e.g., lower perspiration, lower heart rate, lower blood pressure, improved respiration, fewer involuntary movements, etc.—may not be achieved until after the final exercise, e.g., the “Change It” exercise(s), where a patient may receive rational, calm feedback from a virtual friend or therapist about a problematic situation. In some embodiments, voice biomarkers may be used to track emotional states and/or determine intensity values for emotions.

Process 1800 begins at step 1802 in FIG. 18A. At step 1802, a VRCT engine may begin a therapy session for a patient. For instance, the VRCT engine may initiate an exercise to begin therapy, e.g., sitting down with a virtual therapist. In some embodiments, this may include a lobby background or setting. In some embodiments, this may include a nature setting or another peaceful place. In some embodiments, this may include customizing an avatar for a patient, a therapist, and/or a friend character.

At step 1804, the VRCT engine receives and records the patient's first biometric measurements. For instance, in the example data of chart 1820, heart rate (beats per minute) is the selected biometric data and initial reading 1804 is captured at about 160 beats per minute (bpm).

At step 1806, the VRCT engine begins the first exercise(s) of VR Cognitive Therapy, e.g., the “Catch It” exercise(s). In some embodiments, process 800 of FIG. 8 may be used as the “Catch It” exercise. In some embodiments, the VRCT engine may also receive patient input, e.g., as a pre-session emotion input. For instance, a patient may be prompted during an exercise for a situation, thought, emotion, and/or an intensity value. For example, as depicted in patient-reported score 1822 of chart 1820, the patient may report an intensity value of “9” on a scale of 0 to 10 for, e.g., an “anger” emotion. With biometric feedback, for instance, a VRCT engine can monitor whether the patient's body corroborates an intensity value of “9” and determine whether changes happen to the patient during each exercise of the therapy. Such a reading may be set as a baseline for comparison to determine whether a patient lowers such biometric feedback, indicating a less intense emotional response.

At step 1808, the VRCT engine receives and records the patient's second biometric measurements. For instance, in the example data of chart 1820, second reading 1808 is captured at about 150 bpm as the biometric feedback during/after the “Catch It” exercise(s). In some embodiments, this data may be compared to a prior reading to determine whether each exercise is effective. This reading, e.g., second reading at step 1808, may be set as another point for comparison to determine whether a patient lowers such biometric feedback, indicating a less intense emotional response.

At step 1810, the VRCT engine begins the second exercise(s) of VR Cognitive Therapy, e.g., the “Check It” exercise(s). In some embodiments, process 900 of FIG. 9 may be used as the “Check It” exercise.

At step 1812, the VRCT engine receives and records the patient's third biometric measurements. For instance, in the example data of chart 1820, third reading 1812 is captured at about 120 bpm as the biometric feedback during/after the “Check It” exercise(s).

At step 1814, the VRCT engine begins the third exercise(s) of VR Cognitive Therapy, e.g., the “Change It” exercise(s). In some embodiments, process 1000 of FIG. 10 may be used as the “Change It” exercise. In some embodiments, the VRCT engine may also receive patient input, e.g., as a post-session emotion input. For example, a patient may be prompted at the conclusion of an exercise for a situation, thought, emotion, and/or an intensity value. For instance, as depicted in patient-reported score 1824 of chart 1820, the patient may report an intensity value of “3” on a scale of 0 to 10 for, e.g., an “anger” emotion. With biometric feedback, for instance, a VRCT engine can monitor whether the patient's body corroborates an intensity value of “3” and determine whether changes happen to the patient during each exercise of the therapy. In some embodiments, comparison between patient-reported score 1822 and patient-reported score 1824 may indicate if the patient's emotional state is improved and, e.g., that the session was helpful.

At step 1816, the VRCT engine receives and records the patient's fourth biometric measurements. For example, in the sample data of chart 1820, fourth reading 1816 is captured at about 70 bpm as the biometric feedback during/after the “Change It” exercise(s).

At step 1818, the VRCT engine receives and records the patient's final biometric measurements. For instance, in the example data of chart 1820, fifth reading 1818 is captured at about 65 bpm as the biometric feedback after all the exercises. In some embodiments, comparison between patient-reported score 1822 and initial reading 1804, along with comparison of patient-reported score 1824 and readings 1816 or 1818, may indicate if the emotional state of the patient is better than at the start of the session and, e.g., that the session was helpful. In some embodiments, such data may be recorded in a database and tracked from session to session.

In some embodiments, data may be collected to train a neural network to, e.g., categorize emotional states and/or quantify intensity values based on biometric readings. For instance, a model may be trained by a single patient's data and/or a collection of patient data to recognize changes in emotional state. In some embodiments, a trained model may be able to track biometric feedback in a single session and/or over several sessions.

FIG. 18C depicts an illustrative flowchart of a process for comparing biometric measurements for a patient to a patient's input, e.g., during Cognitive Therapy, in accordance with some embodiments of the disclosure. There are many ways to use biometrics with patient input for treating a patient, and process 1850 is one example. Generally, process 1850 of FIG. 18C includes receiving a first biometric measurement, providing a VR-based therapeutic exercise, receiving a second biometric measurement, comparing the biometric measurements, and pausing the exercise and/or alerting the therapist if the comparison of the receiving a first biometric measurements do not reveal the patient's emotional state improving (e.g., getting calmer) during the exercise(s).

Biometrics may be used in conjunction with patient input for, e.g., intensity values of emotions and/or thoughts. In some embodiments, biometrics may be used to determine whether there is a discrepancy between patient-reported feedback and biometrically measured data about the patient, e.g., before, during, and/or after therapy. For example, a patient may report a high intensity value like 9 on a 0 to 10 scale for feeling an emotion, e.g., anxious, but a measure of heart rate, blood pressure, brain activity, and/or perspiration may not corroborate such a high intensity value. A process for determining a discrepancy in patient-reported data may include steps for receiving a patient's biometric measurements, receiving a patient's input, comparing the biometric measurements to the input and determining whether there are any discrepancies in the patient's input. For instance, a patient may not be completely honest in some input, or unaware of subjectivity in his or her input, and a discrepancy in biometric feedback may highlight such an issue.

Some embodiments may utilize a VRCT engine to perform one or more parts of process 1850, e.g., as part of a VR application, stored and executed by one or more of the processors and memory of a headset, server, tablet and/or other device.

At step 1852, a VRCT engine receives a patient's first biometric measurement(s). For instance, FIG. 7 depicts a VR system with exemplary components including several biometric sensors. The biometric sensors measure and record a variety of biometric data including heart rate, respiration, temperature, perspiration, voice/speech (e.g., tone, intensity, pitch, etc.), eye movements, facial movements, mouth and jaw movements, hand and feet movements, neural and brain activities, etc., throughout the Cognitive Therapy session. The biometric data can be used to correlate with the state of emotional wellness of the patient at the start of the Cognitive Therapy session, throughout the exercises, and at the end of the session. In some embodiments, a biometric measurement may be normalized, e.g., to a scale of 0 to 10 or a percentage 0 to 100%

At step 1854, the VRCT engine provides a VR activity and/or exercise to the patient. For instance, the VRCT engine may provide one or more exercises based on the “Catch It,” “Check It,” and/or “Change It” exercises described above and depicted in FIGS. 8-17.

In some embodiments, during an exercise, patient-reported input, such as an intensity value, may be received via audio input, sensor input, accelerometer, mouse, keyboard, touchscreen, etc. For instance, voice input may be received as speech to be converted to text via NLP. A patient may be prompted to say aloud, e.g., an emotion or an intensity score for an emotion. In some embodiments, head position input as a “gaze” may allow aiming and selecting of user interface elements such as buttons, words, numbers, icons, etc. In some embodiments, patient input may be an emotion such as emotions 1222-58 as depicted in scenario 1200 of FIG. 12, e.g., cautious, happy, sad, shy, frustrated, empty, embarrassed, angry, worried, anxious, overwhelmed, hopeful, guilty, nervous, shocked, and others. In some embodiments, an intensity score for one or more emotions may be input by a patient using, e.g., voice and/or gaze selection.

At step 1856, the VRCT engine receives the patient's second biometric measurement(s). Typically, the second biometric will measure the same physical attributes as the first biometric measurement. In some cases, the second biometric may measure a different but similar physical attribute as the first biometric measurement and will be, e.g., normalized for comparison. The biometric measurements may be stored as ledger data. For instance, a ledger may be a data structure where, e.g., patient input is logged. In scenario 1300 of FIG. 13, the VRCT platform displays ledger 1322. A VRCT ledger may be stored in memory as a data structure such as a database, table, spreadsheet, linked list, matrix, etc.

At step 1858, the VRCT engine compares the patient's second biometric measurement to the first biometric measurement to determine whether the patient's emotional state is improving during the provided therapeutic exercise. In some embodiments, a comparison may be between values of the same metric, e.g., (normalized) biometric reading like a blood pressure reading, perspiration measurement, EKG value, etc. For instance, if blood pressure has dropped during the time between the first biometric measurement and the second biometric measurement, it may be determined the patient's emotional state is improving. If brain activity (or facial muscle activity) has safely decreased during the time between the first biometric measurement and the second biometric measurement, it may be determined the emotional state of the patient is improved (e.g., he/she is calmer). In some embodiments, a therapist may be shown a chart, graph, or other pictorial display of such a comparison of biometrics, e.g., over time or over a number of activities.

In some embodiments, biometric measurements may be normalized for comparison. This may be helpful with, e.g., plotting patient-provided intensity values. For instance, a heart rate measurement may be normalized, based on appropriate high and low values for a patient based on age, height, weight, etc. As an example, heart rate values between 60 and 200 beats per minute for a 30-year-old male may be normalized and/or weighted to, e.g., a scale of 0 to 10. Volume or decibel level of voice input may be normalized and attributed to an intensity value of, e.g., 0 to 100. Eye motion or respiration measurements can be correlated to, e.g., a scale of 0 to 10. Measurements with advanced devices like EEG can be correlated to normalized scales, too. Measurements may be personalized and/or normalized over time. In some embodiments, measurements may be input into a trained model to determine whether such biometric data supports or refutes the patient's self-reported emotions and/or intensity levels.

At step 1860, the VRCT engine determines whether the patient's emotional state is improved based on the comparison of the second biometric measurement to the first biometric measurement. In some embodiments, a decrease of values during the time between the first biometric measurement to the second biometric measurement using one or more sensors, such as a temperature measurement, a facial tracker, and a camera and/or light sensor, may identify that a patient is likely less angry. For instance, a measured body temperature above 98.5 degrees (but below, e.g., 100 degrees) may indicate a high emotional intensity for a first biometric measurement but a second biometric measurement of 97.9 degrees may indicate a less high emotional intensity. In some embodiments, a perspiration sensor or an EEG reading may identify that a patient may gradually decline from, e.g., feeling anxious and/or overwhelmed to a lower level like, e.g., cautious and/or worried. Body sensors may collect movement data as first and second biometric values to determine, e.g., if a patient is shaking more or less. For example, a normalized perspiration measurement, e.g., a normalized value of 8.5 on a scale of 0 to 10, may indicate a patient is experiencing acute anxiety for a first biometric measurement, but a second biometric measurement of 4.5 (normalized) may indicate a less high emotional intensity. In some cases, a heart rate reading of above, e.g., 200 beats per minute, may indicate a high intensity of an emotion for a first biometric measurement, but a second biometric measurement of 120 beats per minute may indicate a less high emotional intensity, e.g., the patient feeling calmer. Some biometric feedback tools, like blood pressure monitors and pulse oximeters may also reveal underlying health triggers that could cause and/or complicate reported emotional behavior and intensities.

If, at step 1860, the VRCT engine determines the patient's biometric measurements indicate that the patient's emotional state is improved and/or less intense, then, at step 1868, the VRCT continues to provide the VR activity and/or exercise. For instance, if the exercise is successful in making the patient calmer, the exercises will continue. In some embodiments, a next and/or new exercise may be provided, e.g., upon completion of a task. For instance, after a “Check It” exercise is provided, “Change It” exercise may be provided.

If, at step 1860, the VRCT engine determines the patient's biometric measurements do not indicate that the patient's emotional state is improved and/or less intense, then, at step 1862, the VRCT engine pauses the VR therapy activity and/or exercise. For instance, if the comparison reveals that the second biometric is greater than the first biometric measurement, then the exercise may be paused so the patient can relax or someone can intervene. For example, body sensors may receive input of a body part shaking at a higher rate in the second biometric measurement than the first biometric measurement, which may indicate more nervousness and/or anxiety. If a patient is feeling more of an emotion like anxiety or nervousness (with a relative biometric measurement value) then there might be a need to take a break from the VR activity, change the VR activity, and/or have some type of intervention. As another example, in some embodiments, a voice input loudness measurement may be relatively high (e.g., a 6 on a scale of 0 to 10) as a first biometric measurements but the patient may continue to get louder as a second biometric measurement, which may indicate she is feeling aggravated or provoked by the VRCT activity, environment, and/or character avatars. In some embodiments, a second biometric measurement determined to be less than the first biometric measurement during a comparison may indicate a growth in intensity of emotion. For instance, a measure of lower facial movement or eye movement may indicate an intense focus on an upsetting character or setting within the VR world.

At step 1864, the VRCT engine may alert the supervisor or therapist who is administering the VR therapy that, e.g., the VR therapy exercises/activities may not be helpful. For instance, a therapist device such as a phone, tablet, computer, server, or other network-connected device may be sent an alert and/or notification that the second biometric reading indicates, when compared to the first biometric measurement, that the patient's emotional state is not improving (and may be, in fact, becoming agitated or distressed by the VR exercises).

At step 1866, the VRCT engine may provide an alternative activity, e.g., to help calm or otherwise improve the emotional state for a patient who may have compared biometric data indicating agitation and/or irritation. For instance, in some embodiments, a calming activity such as providing a 3D 360-degree video of nature. In some embodiments, calming music may be played. In some embodiments, meditation exercises may be provided, e.g., activities to help with breathing, concentration, relaxation, or more. In some embodiments, puzzle-based or art-based activities may be provided. In some embodiments, therapy may continue but with a different line of prompts, questioning, exercises, avatars, setting, and/or activities. In some embodiments, the new exercises may be recommended by the VRCT engine. In some embodiments, the new exercises may be recommended by the therapist/supervisor.

In some embodiments, biometric data may be used to supplement and/or adjust patient-reported data. For instance, in some embodiments, biometric values may be used in conjunction with patient input about emotional state and/or intensity values. In some embodiments, biometric data may be used to supplement and/or compare to patient survey data. For instance, a patient may take a survey, such as the PHQ-9. In some cases, surveys such as the PHQ-9 may validate (or contradict) whether a patient's emotional state is improving, e.g., as indicated by biometrics and other feedback. In some embodiments, surveys may indicate whether a patient's input and/or survey responses may not be aligned.

In some embodiments, potential discrepancies in biometric data may be adjusted (or ignored) based on other factors such as the patient's conditions. For instance, motion sensors showing movement indicative of potential nervousness may be discounted if the patient has physical or mental issues causing tremors. Discrepancy data based on blood pressure spikes indicating high intensity emotion might be reduced if the patient is obese. Heart rate data may not be a discrepancy if the patient is an athlete or otherwise in very good shape. Discrepancy data based on sound levels may be weighted differently if the patient has hearing issues. Respiratory illness may affect measurements by a pulse oximeter or respiratory sensors, which could imply a false discrepancy. Someone experiencing eye issues may have decreased eye movement and, accordingly, have a muted eye-movement measurement that may not corroborate a self-reported feeling such as nervousness, anxiety, worry, etc. Someone with chronic depression may experience lower blood pressure measurements.

In some embodiments, the biometric feedback may corroborate self-reported emotions, feelings, and/or intensity values, and the ledger should not be changed. In some embodiments, a patient profile may store past values for self-reported emotions, feelings, intensity values, and other data as well as measurements by biometric sensors and devices. In some embodiments, an indication may be provided to a therapist, e.g., via therapist device, that the patient is accurate, truthful, unbiased, and/or in-tune with his or her emotions and/or intensity of those emotions. In some embodiments, a therapist (or a patient) may be able to view past data collected in order to compare data and examine trends. For instance, charts featuring self-reported data and biometric data may be able to display data that supports or refutes patient input over time. Therapists and doctors may analyze such data to identify if a patient may have a bias in responding in therapy. This data may also be used to train a model such as a neural network to determine whether biometric data supports or contradicts therapy responses, as well as identify potential bias in responses.

In some embodiments, if there is a discrepancy and there is no reason for complete reconciliation of the biometric data, the ledger data may be adjusted. For instance, if a (high) heart rate indicates a higher intensity value for, e.g., anger or anxiety, the ledger data may be adjusted. If a (low) perspiration measurement indicates a lower intensity value for, e.g., anger or anxiety, the ledger data may be adjusted accordingly, too. In some embodiments, self-reported data, e.g., for a health questionnaire, and/or ledger data may be adjusted without displaying the adjustment on the screen to avoid causing additional worry or confusion. For instance, someone self-reporting an intensity value of “8” for anger would probably not like to see an interface indicating that the VRCT engine decreased that intensity value to “6” based upon, e.g., a lower temperature, a lower heart rate, facial expressions, EKG, cameras, and/or other sensors. In some embodiments, the VRCT may provide the adjusted ledger data, e.g., to a therapist device. For example, it may be discouraging to show the patient that her self-reported score or emotion was adjusted. In some embodiments, the VRCT may provide to a therapist, e.g., via a therapist device, an indication that the patient-reported data was inaccurate. For instance, a patient may be exaggerating, underrepresenting, and/or lying about an intensity for an emotion, e.g., saying she feels an intensity level of “9” for anger, while her biometrics indicate a lesser intensity.

FIGS. 19A and 19B are diagrams of an illustrative system, in accordance with some embodiments of the disclosure. A VR system may include a clinician tablet 210, head-mounted display 201 (HMD or headset), small sensors 202, and large sensor 202B. Large sensor 202B may comprise transmitters, in some embodiments, and be referred to as wireless transmitter module 202B. Some embodiments may include sensor chargers, router, router battery, headset controller, power cords, USB cables, and other VR system equipment.

Clinician tablet 210 may be configured to use a touch screen, a power/lock button that turns the component on or off, and a charger/accessory port, e.g., USB-C. For instance, pressing the power button on clinician tablet 210 may power on the tablet or restart the tablet. Once clinician tablet 210 is powered on, a therapist or supervisor may access a user interface and be able to log in; add or select a patient; initialize and sync sensors; select, start, modify, or end a therapy session; view data; and/or log out.

Headset 201 may comprise a power button that turns the component on or off, as well as a charger/accessory port, e.g., USB-C. Headset 201 may also provide visual feedback of virtual reality applications in concert with the clinician tablet and the small and large sensors.

Charging headset 201 may be performed by plugging a headset power cord into the storage dock or an outlet. To turn on headset 201 or restart headset 201, the power button may be pressed. A power button may be on top of the headset. Some embodiments may include a headset controller used to access system settings. For instance, a headset controller may be used only in certain troubleshooting and administrative tasks and not necessarily during patient therapy. Buttons on the controller may be used to control power, connect to headset 201, access settings, or control volume.

The large sensor 202B (e.g., a wireless transmitter module) and small sensors 202 are equipped with mechanical and electrical components that measure position and orientation in physical space and then translate that information to construct a virtual environment. Sensors 202 are turned off and charged when placed in the charging station. Sensors 202 turn on and attempt to sync when removed from the charging station. The sensor charger may act as a dock to store and charge the sensors. In some embodiments, sensors may be placed in sensor bands on a patient. In some embodiments, sensors may be miniaturized and may be placed, mounted, fastened, or pasted directly onto a user.

As shown in illustrative FIG. 19A, various systems disclosed herein consist of a set of position and orientation sensors that are worn by a VR participant, e.g., a therapy patient. These sensors communicate with HMD 201, which immerses the patient in a VR experience. An HMD suitable for VR often comprises one or more displays to enable stereoscopic three-dimensional (3D) images. Such internal displays are typically high-resolution (e.g., 2880×1600 or better) and offer high refresh rate (e.g., 75 Hz). The displays are configured to present 3D images to the patient. VR headsets typically include speakers and microphones for deeper immersion.

HMD 201 is a piece central to immersing a patient in a virtual world in terms of presentation and movement. A headset may allow, for instance, a wide field of view (e.g., 110°) and tracking along six degrees of freedom. HMD 201 may include cameras, accelerometers, gyroscopes, and proximity sensors. VR headsets typically include a processor, usually in the form of a system on a chip (SoC), and memory. In some embodiments, headsets may also use, for example, additional cameras as safety features to help users avoid real-world obstacles. HMD 201 may comprise more than one connectivity option in order to communicate with the therapist's tablet. For instance, an HMD 201 may use an SoC that features WiFi and Bluetooth connectivity, in addition to an available USB connection (e.g., USB Type-C). The USB-C connection may also be used to charge the built-in rechargeable battery for the headset.

A supervisor, such as a health care provider or therapist, may use a tablet, e.g., tablet 210 depicted in FIG. 19A, to control the patient's experience. In some embodiments, tablet 210 runs an application and communicates with a router to cloud software configured to authenticate users and store information. Tablet 210 may communicate with HMD 201 in order to initiate HMD applications, collect relayed sensor data, and update records on the cloud servers. Tablet 210 may be stored in the portable container and plugged in to charge, e.g., via a USB plug.

In some embodiments, such as depicted in FIG. 19B, sensors 202 are placed on the body in particular places to measure body movement and relay the measurements for translation and animation of a VR avatar. Sensors 202 may be strapped to a body via bands 205. In some embodiments, each patient may have her own set of bands 205 to minimize hygiene issues.

A wireless transmitter module (WTM) 202B may be worn on a sensor band 205B that is laid over the patient's shoulders. WTM 202B sits between the patient's shoulder blades on their back. Wireless sensor modules 202 (e.g., sensors or WSMs) are worn just above each elbow, strapped to the back of each hand, and on a pelvis band that positions a sensor adjacent to the patient's sacrum on their back. In some embodiments, each WSM communicates its position and orientation in real-time with an HMD Accessory located on the HMD. Each sensor 202 may learn its relative position and orientation to the WTM, e.g., via calibration.

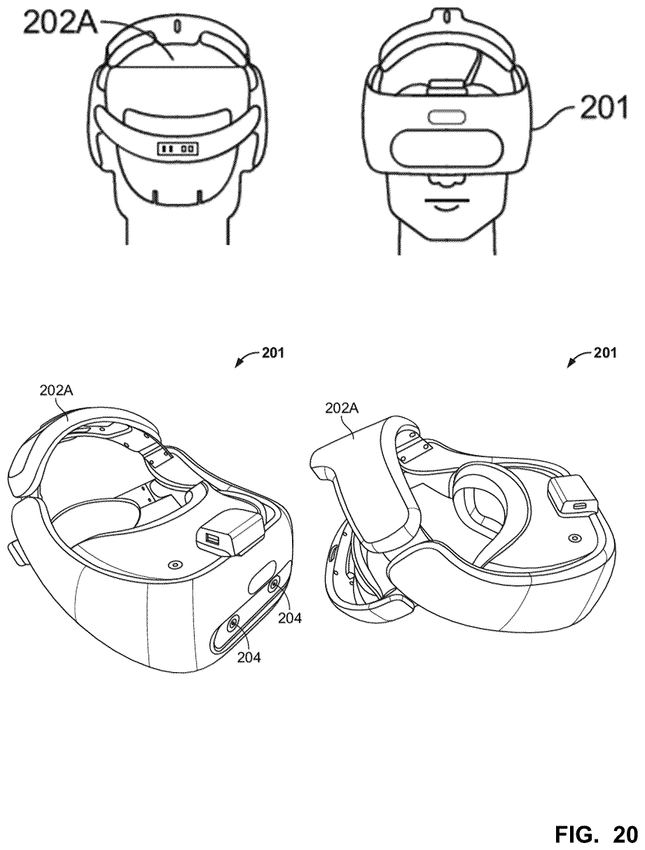

As depicted in FIG. 20, the HMD accessory may include a sensor 202A that may allow it to learn its position relative to WTM 202B, which then allows the HMD to know where in physical space all the WSMs and WTM are located. In some embodiments, each sensor 202 communicates independently with the HMD accessory which then transmits its data to HMD 201, e.g., via a USB-C connection. In some embodiments, each sensor 202 communicates its position and orientation in real-time with WTM 202B, which is in wireless communication with HMD 201. In some embodiments HMD 201 may be connected to input supplying other data such as biometric feedback data. For instance, in some cases, the VR system may include heart rate monitors, electrical signal monitors, e.g., electrocardiogram (EKG), eye movement tracking, brain monitoring with Electroencephalogram (EEG), pulse oximeter monitors, temperature sensors, blood pressure monitors, respiratory monitors, light sensors, cameras, sensors, and other biometric devices. Biometric feedback, along with other performance data, can indicate more subtle changes to the patient's body or physiology as well as mental state, e.g., when a patient is stressed, comfortable, distracted, tired, over-worked, under-worked, over-stimulated, confused, overwhelmed, excited, engaged, disengaged, and more. In some embodiments, such devices measuring biometric feedback may be connected to the HMD and/or the supervisor tablet via USB, Bluetooth, Wi-Fi, radio frequency, and other mechanisms of networking and communication.

A VR environment rendering engine on HMD 201 (sometimes referred to herein as a “VR application”), such as the Unreal Engine™, uses the position and orientation data to create an avatar that mimics the patient's movement.

A patient or player may “become” their avatar when they log in to a virtual reality activity. When the player moves their body, they see their avatar move accordingly. Sensors in the headset may allow the patient to move the avatar's head, e.g., even before body sensors are placed on the patient. A system that achieves consistent high-quality tracking facilitates the patient's movements to be accurately mapped onto an avatar.

Sensors 202 may be placed on the body, e.g., of a patient by a therapist, in particular locations to sense and/or translate body movements. The system can use measurements of position and orientation of sensors placed in key places to determine movement of body parts in the real world and translate such movement to the virtual world. In some embodiments, a VR system may collect performance data for therapeutic analysis of a patient's movements and range of motion.

In some embodiments, systems and methods of the present disclosure may use electromagnetic tracking, optical tracking, infrared tracking, accelerometers, magnetometers, gyroscopes, myoelectric tracking, other tracking techniques, or a combination of one or more of such tracking methods. The tracking systems may be parts of a computing system as disclosed herein. The tracking tools may exist on one or more circuit boards within the VR system (see FIG. 21) where they may monitor one or more users to perform one or more functions such as capturing, analyzing, and/or tracking a subject's movement. In some cases, a VR system may utilize more than one tracking method to improve reliability, accuracy, and precision.

FIG. 21 depicts an illustrative arrangement for various elements of a system, e.g., an HMD and sensors of FIGS. 19A-B and FIG. 20. The arrangement includes one or more printed circuit boards (PCBs). In general terms, the elements of this arrangement track, model, and display a visual representation of the participant (e.g., a patient avatar) in the VR world by running software including the aforementioned VR application of HMD 201.

The arrangement shown in FIG. 21 includes one or more sensors 992, processors 960, graphic processing units (GPUs) 920, video encoder/video codec 940, sound cards 946, transmitter modules 990, network interfaces 980, and light emitting diodes (LEDs) 969. These components may be housed on a local computing system or may be remote components in wired or wireless connection with a local computing system (e.g., a remote server, a cloud, a mobile device, a connected device, etc.). Connections between components may be facilitated by one or more buses, such as bus 914, bus 934, bus 948, bus 984, and bus 964 (e.g., peripheral component interconnects (PCI) bus, PCI-Express bus, or universal serial bus (USB)). With such buses, the computing environment may be capable of integrating numerous components, numerous PCBs, and/or numerous remote computing systems.

One or more system management controllers, such as system management controller 912 or system management controller 932, may provide data transmission management functions between the buses and the components they integrate. For instance, system management controller 912 provides data transmission management functions between bus 914 and sensors 992. System management controller 932 provides data transmission management functions between bus 934 and GPU 920. Such management controllers may facilitate the arrangements orchestration of these components that may each utilize separate instructions within defined time frames to execute applications. Network interface 980 may include an ethernet connection or a component that forms a wireless connection, e.g., 802.11b, g, a, or n connection (WiFi), to a local area network (LAN) 987, wide area network (WAN) 983, intranet 985, or internet 981. Network controller 982 provides data transmission management functions between bus 984 and network interface 980.

A device may receive content and data via input/output (hereinafter “I/O”) path. I/O path may provide content (e.g., content available over a local area network (LAN) or wide area network (WAN), and/or other content) and data to control circuitry 1204, which includes processing circuitry 1206 and storage 1208. Control circuitry may be used to send and receive commands, requests, and other suitable data using I/O path. I/O path may connect control circuitry (and processing circuitry) to one or more communications paths. I/O functions may be provided by one or more of these communications paths.

Control circuitry may be based on any suitable processing circuitry such as processing circuitry. As referred to herein, processing circuitry should be understood to mean circuitry based on one or more microprocessors, microcontrollers, digital signal processors, programmable logic devices, field-programmable gate arrays (FPGAs), application-specific integrated circuits (ASICs), etc., and may include a multi-core processor (e.g., dual-core, quad-core, hexa-core, or any suitable number of cores). In some embodiments, processing circuitry may be distributed across multiple separate processors or processing units, for example, multiple of the same type of processing units (e.g., two Intel Core i7 processors) or multiple different processors (e.g., an Intel Core i5 processor and an Intel Core i7 processor). In some embodiments, control circuitry executes instructions for receiving streamed content and executing its display, such as executing application programs that provide interfaces for content providers to stream and display content on a display.

Control circuitry may thus include communications circuitry suitable for communicating with a content provider server or other networks or servers. Communications circuitry may include a cable modem, an integrated services digital network (ISDN) modem, a digital subscriber line (DSL) modem, a telephone modem, Ethernet card, or a wireless modem for communications with other equipment, or any other suitable communications circuitry. Such communications may involve the Internet or any other suitable communications networks or paths. In addition, communications circuitry may include circuitry that enables peer-to-peer communication of user equipment devices, or communication of user equipment devices in locations remote from each other.

Processor(s) 960 and GPU 920 may execute a number of instructions, such as machine-readable instructions. The instructions may include instructions for receiving, storing, processing, and transmitting tracking data from various sources, such as electromagnetic (EM) sensors 993, optical sensors 994, infrared (IR) sensors 997, inertial measurement units (IMUs) sensors 995, and/or myoelectric sensors 996. The tracking data may be communicated to processor(s) 960 by either a wired or wireless communication link, e.g., transmitter 990. Upon receiving tracking data, processor(s) 960 may execute an instruction to permanently or temporarily store the tracking data in memory 962 such as, e.g., random access memory (RAM), read only memory (ROM), cache, flash memory, hard disk, or other suitable storage component. Memory may be a separate component, such as memory 968, in communication with processor(s) 960 or may be integrated into processor(s) 960, such as memory 962, as depicted.

Memory may be an electronic storage device provided as storage that is part of control circuitry. As referred to herein, the phrase “electronic storage device” or “storage device” should be understood to mean any device for storing electronic data, computer software, or firmware, such as random-access memory, read-only memory, hard drives, optical drives, digital video disc (DVD) recorders, compact disc (CD) recorders, BLU-RAY disc (BD) recorders, BLU-RAY 3D disc recorders, digital video recorders (DVR, sometimes called a personal video recorder, or PVR), solid state devices, quantum storage devices, gaming consoles, gaming media, or any other suitable fixed or removable storage devices, and/or any combination of the same. Storage may be used to store various types of content described herein as well as media guidance data described above. Nonvolatile memory may also be used (e.g., to launch a boot-up routine and other instructions). Cloud-based storage may be used to supplement storage or instead of storage.

Storage may also store instructions or code for an operating system and any number of application programs to be executed by the operating system. In operation, processing circuitry retrieves and executes the instructions stored in storage, to run both the operating system and any application programs started by the user. The application programs can include one or more voice interface applications for implementing voice communication with a user, and/or content display applications which implement an interface allowing users to select and display content on display or another display.

Processor(s) 960 may also execute instructions for constructing an instance of virtual space. The instance may be hosted on an external server and may persist and undergo changes even when a participant is not logged in to said instance. In some embodiments, the instance may be participant-specific, and the data required to construct it may be stored locally. In such an embodiment, new instance data may be distributed as updates that users download from an external source into local memory. In some exemplary embodiments, the instance of virtual space may include a virtual volume of space, a virtual topography (e.g., ground, mountains, lakes), virtual objects, and virtual characters (e.g., non-player characters “NPCs”). The instance may be constructed and/or rendered in 2D or 3D. The rendering may offer the viewer a first-person or third-person perspective. A first-person perspective may include displaying the virtual world from the eyes of the avatar and allowing the patient to view body movements from the avatar's perspective. A third-person perspective may include displaying the virtual world from, for example, behind the avatar to allow someone to view body movements from a different perspective. The instance may include properties of physics, such as gravity, magnetism, mass, force, velocity, and acceleration, which cause the virtual objects in the virtual space to behave in a manner at least visually similar to the behaviors of real objects in real space.

Processor(s) 960 may execute a program (e.g., the Unreal Engine or VR applications discussed above) for analyzing and modeling tracking data. For instance, processor(s) 960 may execute a program that analyzes the tracking data it receives according to algorithms described above, along with other related pertinent mathematical formulas. Such a program may incorporate a graphics processing unit (GPU) 920 that is capable of translating tracking data into 3D models. GPU 920 may utilize shader engine 928, vertex animation 924, and linear blend skinning algorithms. In some instances, processor(s) 960 or a CPU may at least partially assist the GPU in making such calculations. This allows GPU 920 to dedicate more resources to the task of converting 3D scene data to the projected render buffer. GPU 920 may refine the 3D model by using one or more algorithms, such as an algorithm learned on biomechanical movements, a cascading algorithm that converges on a solution by parsing and incrementally considering several sources of tracking data, an inverse kinematics (IK) engine 930, a proportionality algorithm, and other algorithms related to data processing and animation techniques. After GPU 920 constructs a suitable 3D model, processor(s) 960 executes a program to transmit data for the 3D model to another component of the computing environment (or to a peripheral component in communication with the computing environment) that is capable of displaying the model, such as display 950.

In some embodiments, GPU 920 transfers the 3D model to a video encoder or a video codec 940 via a bus, which then transfers information representative of the 3D model to a suitable display 950. The 3D model may be representative of a virtual entity that can be displayed in an instance of virtual space, e.g., an avatar. The virtual entity is capable of interacting with the virtual topography, virtual objects, and virtual characters within virtual space. The virtual entity is controlled by a user's movements, as interpreted by sensors 992 communicating with the system. Display 950 may display a Patient View. The patient's real-world movements are reflected by the avatar in the virtual world. The virtual world may be viewed in the headset in 3D and monitored on the tablet in two dimensions. In some embodiments, the VR world is an activity that provides feedback and rewards based on the patient's ability to complete activities. Data from the in-world avatar is transmitted from the HMD to the tablet to the cloud, where it is stored for later analysis. An illustrative architectural diagram of such elements in accordance with some embodiments is depicted in FIG. 22.

A VR system may also comprise display 970, which is connected to the computing environment via transmitter 972. Display 970 may be a component of a clinician tablet. For instance, a supervisor or operator, such as a therapist, may securely log in to a clinician tablet, coupled to the system, to observe and direct the patient to participate in various activities and adjust the parameters of the activities to best suit the patient's ability level. Display 970 may depict a view of the avatar and/or replicate the view of the HMD.

In some embodiments, HMD 201 may be the same as or similar to HMD 1010 in FIG. 22. In some embodiments, HMD 1010 runs a version of Android that is provided by HTC (e.g., a headset manufacturer) and the VR application is an Unreal application, e.g., Unreal Application 1016, encoded in an Android package (.apk). The .apk comprises a set of custom plugins: WVR, WaveVR, SixenseCore, SixenseLib, and MVICore. The WVR and WaveVR plugins allow the Unreal application to communicate with the VR headset's functionality. The SixenseCore, SixenseLib, and MVICore plugins allow Unreal Application 1016 to communicate with the HMD accessory and sensors that communicate with the HMD via USB-C. The Unreal Application comprises code that records the position and orientation (PnO) data of the hardware sensors and translates that data into a patient avatar, which mimics the patient's motion within the VR world. An avatar can be used, for example, to infer and measure the patient's real-world range of motion. The Unreal application of the HMD includes an avatar solver as described, for example, below.

The clinician operator device, clinician tablet 1020, runs a native application (e.g., Android application 1025) that allows an operator such as a therapist to control a patient's experience. Cloud server 1050 includes a combination of software that manages authentication, data storage and retrieval, and hosts the user interface, which runs on the tablet. This can be accessed by tablet 1020. Tablet 1020 has several modules.

As depicted in FIG. 22, the first part of tablet software is a mobile device management (MDM) 1024 layer, configured to control what software runs on the tablet, enable/disable the software remotely, and remotely upgrade the tablet applications.

The second part is an application, e.g., Android Application 1025, configured to allow an operator to control the software of HMD 1010. In some embodiments, the application may be a native application. A native application, in turn, may comprise two parts, e.g., (1) socket host 1026 configured to receive native socket communications from the HMD and translate that content into web sockets, e.g., web sockets 1027, that a web browser can easily interpret; and (2) a web browser 1028, which is what the operator sees on the tablet screen. The web browser may receive data from the HMD via the socket host 1026, which translates the HMD's native socket communication 1018 into web sockets 1027, and it may receive UI/UX information from a file server 1052 in cloud 1050. Tablet 1020 comprises web browser 1028, which may incorporate a real-time 3D engine, such as Babylon.js, using a JavaScript library for displaying 3D graphics in web browser 1028 via HTML5. For instance, a real-time 3D engine, such as Babylon.js, may render 3D graphics, e.g., in web browser 1028 on clinician tablet 1020, based on received skeletal data from an avatar solver in the Unreal Engine 1016 stored and executed on HMD 1010. In some embodiments, rather than Android Application 1026, there may be a web application or other software to communicate with file server 1052 in cloud 1050. In some instances, an application of Tablet 1020 may use, e.g., Web Real-Time Communication (WebRTC) to facilitate peer-to-peer communication without plugins, native apps, and/or web sockets.

The cloud software, e.g., cloud 1050, has several different, interconnected parts configured to communicate with the tablet software: authorization and API server 1062, GraphQL server 1064, and file server (static web host) 1052.

In some embodiments, authorization and API server 1062 may be used as a gatekeeper. For example, when an operator attempts to log in to the system, the tablet communicates with the authorization server. This server ensures that interactions (e.g., queries, updates, etc.) are authorized based on session variables such as operator's role, the health care organization, and the current patient. This server, or group of servers, communicates with several parts of the system: (a) a key value store 1054, which is a clustered session cache that stores and allows quick retrieval of session variables; (b) a GraphQL server 1064, as discussed below, which is used to access the back-end database in order to populate the key value store, and also for some calls to the application programming interface (API); (c) an identity server 1056 for handling the user login process; and (d) a secrets manager 1058 for injecting service passwords (relational database, identity database, identity server, key value store) into the environment in lieu of hard coding.

When the tablet requests data, it will communicate with the GraphQL server 1064, which will, in turn, communicate with several parts: (1) the authorization and API server 1062; (2) the secrets manager 1058, and (3) a relational database 1053 storing data for the system. Data stored by the relational database 1053 may include, for instance, profile data, session data, application data, activity performance data, and motion data.

In some embodiments, profile data may include information used to identify the patient, such as a name or an alias. Session data may comprise information about the patient's previous sessions, as well as, for example, a “free text” field into which the therapist can input unrestricted text, and a log 1055 of the patient's previous activity. Logs 1055 are typically used for session data and may include, for example, total activity time, e.g., how long the patient was actively engaged with individual activities; activity summary, e.g., a list of which activities the patient performed, and how long they engaged with each on; and settings and results for each activity. Activity performance data may incorporate information about the patient's progression through the activity content of the VR world. Motion data may include specific range-of-motion (ROM) data that may be saved about the patient's movement over the course of each activity and session, so that therapists can compare session data to previous sessions' data.

In some embodiments, file server 1052 may serve the tablet software's website as a static web host.

Cloud server 1050 may also include one or more systems for implementing processes of voice processing in accordance with some embodiments of the disclosure. For instance, such a system may perform voice identification/differentiation, determination of interrupting and supplemental comments, and processing of voice queries. A computing device 1100 may be in communication with an automated speech recognition (ASR) server 1057 through, for example, a communications network. ASR server 1057 may also be in electronic communication with natural language processing (NLP) server 1059 also through, for example, a communications network. ASR server 1057 and/or NLP server 1059 may be in communication with one or more computing devices running a user interface, such as a voice assistant, voice interface allowing for voice-based communication with a user, or an electronic content display system for a user. Examples of such computing devices are a smart home assistant similar to a Google Home® device or an Amazon® Alexa® or Echo® device, a smartphone or laptop computer with a voice interface application for receiving and broadcasting information in voice format, a set-top box or television running a media guide program or other content display program for a user, or a server executing a content display application for generating content for display to a user. ASR server 1057 may be any server running an ASR application. NLP server 1059 may be any server programmed to process one or more voice inputs in accordance with some embodiments of the disclosure, and to process voice queries with the ASR server 1057. In some embodiments, one or more of ASR server 1057 and NLP server 1059 may be components of cloud server 1050 depicted in FIG. 22.

While the foregoing discussion describes exemplary embodiments of the present invention, one skilled in the art will recognize from such discussion, the accompanying drawings, and the claims, that various modifications can be made without departing from the spirit and scope of the invention. Therefore, the illustrations and examples herein should be construed in an illustrative, and not a restrictive sense. The scope and spirit of the invention should be measured solely by reference to the claims that follow.

1. A method of providing Cognitive Therapy in a virtual reality (VR) platform, the method comprising:
providing, via a therapist avatar in the VR platform, a first prompt requesting a first description of a patient situation associated with an emotional state of a patient;
receiving, via patient input, the first description of the patient situation;
providing, via the therapist avatar in the VR platform, a second prompt requesting from the patient an initial thought for the patient situation;
receiving, via patient input, the initial thought;
providing, via the therapist avatar in the VR platform, a third prompt requesting evidence supporting the initial thought and evidence refuting the initial thought;
receiving, via patient input, evidence supporting the initial thought and evidence refuting the initial thought;
providing, via a friend avatar in the VR platform, a friend situation description of the friend avatar that recites at least a portion of the received first description of the patient situation;
providing, via the therapist avatar in the VR platform, a fourth prompt requesting a patient response to the friend situation description;
providing, via a user interface in the VR platform, a suggested response for the patient to tell the virtual friend comprising evidence refuting the initial thought from a second-person point of view;
receiving, via patient input, the patient response to the friend situation description;
providing, via the therapist avatar in the VR platform, a fifth prompt requesting the patient to provide a second description of the patient situation to the friend avatar;
receiving, via patient input, the second description of the patient situation; and
providing, via the friend avatar in the VR platform, a friend response to the received second description of the patient situation that recites at least a portion of the patient response.

2. The method of claim 1, wherein the first description of the patient situation comprises a first intensity value for associated with an emotional state, the method further comprising:
providing, via the virtual reality platform, a sixth prompt requesting a second intensity value for the emotional state; and
comparing the second intensity value to the first intensity value;
in response to determining the second intensity value is less than the first intensity value, providing, via the virtual reality platform, a congratulatory message based on the second intensity value; and
in response to determining the second intensity value is not less than the first intensity value, providing, via the virtual reality platform, an appreciation message based on the second intensity value.

3. The method of claim 1, wherein the therapist avatar in the VR platform is created by:
receiving a physical input parameter of a virtual therapist.

4. The method of claim 3, wherein the received physical input parameter of the virtual therapist comprises:
an age of a virtual therapist;
a gender of the virtual therapist;
a height of the virtual therapist;
a weight of the virtual therapist;
a hairstyle of the virtual therapist; and
a clothing style of the virtual therapist; and
the method further comprises rendering, in the virtual reality platform, the virtual therapist as the therapist avatar based on the age, gender, height, weight, hairstyle, and clothing style.

5. The method of claim 1, wherein the suggested response is presented with the received evidence refuting the initial thought in a ledger user interface element.

6. The method of claim 5, wherein the ledger user interface element further comprises the first description of the patient situation and evidence supporting the initial thought.

7. (canceled)

8. The method of claim 1, wherein the receiving, via patient input, the first description of the patient situation further comprises:
providing, by the VR platform, a prompt requesting an intensity score for the emotional state of a patient;
receiving an input from the patient, the input comprising the intensity score;
normalizing the intensity score;
receiving a biometric measurement for a patient related to a condition of the patient for the emotion;
normalizing the biometric measurement;
determining a discrepancy between the normalized biometric measurement and the normalized intensity score; and
recording the discrepancy between the normalized biometric measurement and the normalized intensity score.

9. The method of claim 8 further comprising:
in response to determining the discrepancy between the normalized biometric measurement and the normalized intensity score is greater than a predetermined threshold:
(a) adjusting the intensity score based on the normalized biometric measurement; and
(b) providing the adjusted intensity score for display; and
in response to determining the discrepancy between the normalized biometric measurement and the normalized intensity score is not greater than a predetermined threshold, providing the intensity score, without adjustment, for display.

10. The method of claim 1, wherein receiving, via patient input, the first description of the patient situation comprises:
accessing, by the VR platform, a plurality of emotions;
generating, via the user interface in the VR platform, for display a user interface (UI) element for each of the plurality of emotions;
receiving, via patient input, a selection of a UI element corresponding to a selected emotion from the plurality of emotions;
providing a prompt requesting a first intensity value for the selected emotion;
receiving input comprising the first intensity value for the selected emotion; and
storing in a ledger data structure the first intensity value and the selected emotion.

11. The method of claim 1, wherein the friend avatar in the VR platform is created by:
receiving a physical input parameter of a virtual friend.

12. The method of claim 11, wherein the received physical input parameter of the virtual friend comprises:
an age of the virtual friend;
a gender of the virtual friend;
a height of the virtual friend;
a weight of the virtual friend;
a hairstyle of the virtual friend; and
a clothing style of the virtual friend; and
the method further comprises rendering, in the virtual reality platform, the virtual friend as the friend avatar based on the age, gender, height, weight, hairstyle, and clothing style.

13. A method of providing therapy to a patient via a virtual reality (VR) platform, the method comprising:
receiving a first biometric value from a biometric device in communication with the VR platform, the first biometric value measuring the patient as a baseline before a therapeutic exercise;
providing, by the VR platform, a therapeutic exercise to the patient;
receiving a second biometric value from the biometric device, the second biometric value measuring the patient during the therapeutic exercise;
comparing the second biometric value to the first biometric value to determine whether the patient has improvement in an emotional state from the therapeutic exercise;
in response to determining the patient has no improvement in the emotional state from the therapeutic exercise, pausing the therapeutic exercise and providing an alert; and
in response to determining the patient has improvement in the emotional state from the therapeutic exercise, continuing to provide the therapeutic exercise.

14. The method of claim 13, wherein comparing the second biometric value to the first biometric value to determine whether the patient has improvement in the emotional state from the therapeutic exercise comprises determining whether the second biometric value is less than at least one of the following: the first biometric value and a predetermined threshold value.

15. The method of claim 13, wherein comparing the second biometric value to the first biometric value to determine whether the patient has improvement in the emotional state from the therapeutic exercise comprises determining whether the second biometric value is not greater than a predetermined percentage of the first biometric value.

16. The method of claim 13, wherein comparing the second biometric value to the first biometric value to determine whether the patient has improvement in the emotional state from the therapeutic exercise comprises:
normalizing the first biometric value;
normalizing the second biometric value; and
determining whether the second biometric value is less than the first biometric value.

17. (canceled)

18. The method of claim 13 further comprising providing for display patient data comprising the first biometric value and the second biometric value to a therapist device as at least one of the following: a chart, a table, a graph, a hierarchy, and a list.

19. The method of claim 13 further comprising:
providing, by the VR platform, a second therapeutic exercise to the patient;
receiving a third biometric value from the biometric device, the third biometric value measuring the patient during the second therapeutic exercise;
comparing the third biometric value to the second biometric value to determine whether the patient has improvement in the emotional state from the second therapeutic exercise;
in response to determining the patient has no improvement in the emotional state from the second therapeutic exercise, pausing the second therapeutic exercise and providing an alert; and
in response to determining the patient has improvement in the emotional state from the second therapeutic exercise, continuing to provide the second therapeutic exercise.

20. The method of claim 13, wherein the biometric measurement is selected from one of the following: heart rate, respiration, temperature, perspiration, voice tone, voice intensity, voice pitch, eye movement, facial movement, mouth movement, jaw movement, hand movement, feet movement, neural activities, and brain activities.

21. The method of claim 13, wherein the biometric measurement is transmitted from at least one of the following: an eye movement tracker, an electroencephalogram (EEG), a temperature sensor, a respiratory monitor, a microphone, a facial reflexive movement tracker, a facial expression monitor, an electrocardiogram (EKG), a blood pressure monitor, a perspiration sensor, a pulse oximeter monitor, a camera, and a light sensor.

22. The method of claim 13 further comprising:
receiving an intensity value input from the patient during the therapeutic exercise;
normalizing the intensity value;
normalizing the second biometric value; and
determining whether there is a discrepancy between the second biometric value and the intensity value;
in response to determining a discrepancy exists, adjusting the intensity value based on the second biometric value; and
in response to determining there is no discrepancy, continuing to provide the therapeutic exercise.

23.-46. (canceled)
